(19) United States
(12) Patent Application Publication
(10) Pub. No.: US 2026/0242685 A1
(43) Pub. Date: Aug. 20, 2026

(54) TETRAMERS OF 1-OCTENE AND POLYALPHAOLEFIN COMPOSITIONS PRODUCED THEREFROM

(71) Applicant: Chevron Phillips Chemical Company LP, The Woodlands, TX (US)

(72) Inventors: Orson L. Sydora, Kingwood, TX (US); Thomas J. Malinski, The Woodlands, TX (US)

(21) Appl. No.: 19/540,857

(22) Filed: Feb. 16, 2026

Related U.S. Application Data
(60) Provisional application No. 63/759,616, filed on Feb. 18, 2025.

(51) Int. Cl. C10M 105/04; C07C 2/08; C07C 9/22

(52) U.S. Cl. CPC C10M 105/04; C07C 2/08; C07C 9/22; C10M 2203/003

(57) ABSTRACT

Processes for producing a 1-octene tetramer (C₃₂) product include the steps of contacting a first catalyst composition with an olefin feedstock comprising at least 50 wt. % of 1-octene under first oligomerization conditions to produce a first oligomer product comprising a C₁₆ vinylidene dimer, followed by separating the C₁₆ vinylidene dimer from the first oligomer product, and then contacting the C₁₆ vinylidene dimer with a second catalyst composition under second oligomerization conditions to produce a second oligomer product comprising at least 50 wt. % of C₃₂ olefins. Polyalphaolefins can subsequently be produced by separating the C₃₂ olefins from the second oligomer product and hydrogenating the C₃₂ olefins to form the polyalphaolefin product. Polyalphaolefins produced from the C₃₂ olefins have an advantageous combination of kinematic viscosity, pour point, flash point, and viscosity index.

### REFERENCE TO RELATED APPLICATION

This application claims the benefit of U.S. Provisional Patent Application No. 63/759,616, filed on Feb. 18, 2025, the disclosure of which is incorporated herein by reference in its entirety.

### TECHNICAL FIELD

The present disclosure generally relates to processes for oligomerizing 1-octene, which involve the initial formation of a C₁₆ vinylidene dimer from an olefin feedstock comprising 1-octene, followed by the oligomerization of the C₁₆ vinylidene dimer to produce C₃₂ olefins. Low viscosity polyalphaolefins can be produced from the C₃₂ olefins for use in lubricant formulations and other related end-use applications.

### BACKGROUND

The superior low temperature lubrication properties of low viscosity polyalphaolefins (PAOs) mainly derive from their feedstock, typically 1-decene, and the cationic mechanism utilized in their production. However, the reliance on 1-decene as a feedstock poses challenges related to its availability and cost. Thus, there is a need for oligomerization processes that utilize an alternative feedstock that can produce polyalphaolefin products that retain the superior low-temperature performance properties and other favorable characteristics present in PAOs derived from 1-decene. Accordingly, it is to these ends that the present invention is generally directed.

### SUMMARY

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Processes for oligomerizing 1-octene to produce C₃₂ olefins are disclosed herein. In accordance with an aspect of the present invention, one such oligomerization process can comprise (i) contacting a first catalyst composition with an olefin feedstock comprising at least 50 wt. % of 1-octene under first oligomerization conditions to produce a first oligomer product comprising a C₁₆ vinylidene dimer, (ii) separating the C₁₆ vinylidene dimer from the first oligomer product, and (iii) contacting the C₁₆ vinylidene dimer with a second catalyst composition under second oligomerization conditions to produce a second oligomer product comprising at least 50 wt. % of C₃₂ olefins. Unexpectedly, the disclosed oligomerization processes can achieve high conversion efficiency in both oligomerization steps.

Additionally, processes to produce a polyalphaolefin (PAO) are disclosed herein. One such process can comprise (I) contacting a first catalyst composition with an olefin feedstock comprising at least 50 wt. % of 1-octene under first oligomerization conditions to produce a first oligomer product comprising a C₁₆ vinylidene dimer, (II) separating the C₁₆ vinylidene dimer from the first oligomer product, (III) contacting the C₁₆ vinylidene dimer with a second catalyst composition under second oligomerization conditions to produce a second oligomer product comprising at least 50 wt. % of C₃₂ olefins, (IV) separating the C₃₂ olefins from the second oligomer product, and (V) hydrogenating the C₃₂ olefins to form the polyalphaolefin. The disclosed processes to produce a polyalphaolefin unexpectedly result in low viscosity polyalphaolefins with beneficial combinations of kinematic viscosity, pour point, flash point, and viscosity index.

Other aspects are directed to low viscosity polyalphaolefins (PAOs) (also referred to as polyalphaolefin compositions) that can be utilized in lubricant formulations and other related end-use applications. A first polyalphaolefin consistent with this disclosure can have a 100° C. kinematic viscosity (KV100) in a range from 3.5 to 5 cSt, a pour point in a range from −90 to −50° C., and a viscosity index in a range from 90 to 120.

A second polyalphaolefin provided herein can comprise at least 70 wt. % of C₃₂ alkanes (hydrogenated 1-octene tetramers) and can have a 100° C. kinematic viscosity (KV100) in a range from 3.5 to 5 cSt, a pour point in a range from −90 to −50° C., and a flash point in a range from 200 to 300° C.

A third polyalphaolefin provided herein can comprise at least 70 wt. % of C₃₂ alkanes (hydrogenated 1-octene tetramers) and can have a 100° C. kinematic viscosity (KV100) in a range from 3.5 to 5 cSt, a pour point in a range from −90 to −50° C., and a viscosity index in a range from 90 to 120.

A fourth polyalphaolefin provided herein can comprise at least 70 wt. % of C₃₂ alkanes (hydrogenated 1-octene tetramers) and can have a 100° C. kinematic viscosity (KV100) in a range from 3.5 to 5 cSt. For the fourth polyalphaolefin, the C₃₂ alkanes can comprise at least 25 wt. % of a compound having the structure:

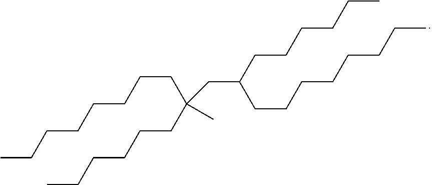 [SMILES: CCCCCCCCC(CCCCCC)CC(C)(CCCCCC)CCCCCCCC]

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the examples and detailed description.

### BRIEF DESCRIPTION OF THE FIGURES

The following figures form part of the present specification and are included to further demonstrate certain aspects of the present invention. The invention may be better understood by reference to these figures in combination with the detailed description.

FIG. 1 presents a portion of a gas chromatography plot of the unhydrogenated 1-octene tetramer (C₃₂) of Example 9.

FIG. 2 presents a portion of a gas chromatography plot of the hydrogenated 1-octene tetramer (C₃₂) PAO of Example 10.

FIG. 3 presents a ¹H NMR plot of the hydrogenated 1-octene tetramer (C₃₂) PAO of Example 10.

FIG. 4 presents a ¹³C NMR plot of the hydrogenated 1-octene tetramer (C₃₂) PAO of Example 10.

While the inventions disclosed herein are susceptible to various modifications and alternative forms, only a few specific aspects have been shown by way of example in the drawings and described in detail below. The figures and detailed description of specific aspects are not intended to limit the breadth or scope of the inventive concepts or the appended claims in any manner. Rather, the figures and detailed description are provided to illustrate the inventive concepts to a person of ordinary skill in the art and to enable such person to make and use the inventive concepts.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compositions, processes, or methods described herein are contemplated with or without explicit description of the particular combination. Additionally, unless explicitly recited otherwise, any aspect or feature disclosed herein can be combined to describe inventive compositions, processes, or methods consistent with the present disclosure.

In this disclosure, while compositions and processes are described in terms of “comprising” various components or steps, the compositions and processes also can “consist essentially of” or “consist of” the various components or steps, unless stated otherwise.

The terms “a,” “an,” and “the” are intended to include plural alternatives, e.g., at least one. For instance, the disclosure of “a catalyst” is meant to encompass one catalyst, or mixtures or combinations of more than one catalyst, unless otherwise specified.

The term “contacting” is used herein to describe compositions and processes/methods in which the materials are contacted together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique. Herein, “contacting” an olefin material with a suitable catalyst can result in an oligomer product.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, transition metals for Group 3-12 elements, and halogens for Group 17 elements.

The term “hydrocarbon” refers to a compound containing only carbon and hydrogen. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “alkane” refers to a saturated hydrocarbon compound.

The term “olefin” refers to hydrocarbons that have at least one carbon-carbon double bond that is not part of an aromatic ring or an aromatic ring system. The term “olefin” includes aliphatic and aromatic, cyclic and acyclic, and/or linear and branched hydrocarbons having at least one carbon-carbon double bond that is not part of an aromatic ring or ring system unless specifically stated otherwise. Olefins having only one, only two, only three, etc., carbon-carbon double bonds can be identified by use of the term “mono,” “di,” “tri,” etc., within the name of the olefin. The olefins can be further identified by the position of the carbon-carbon double bond(s).

The term “alpha olefin” as used herein refers to any olefin that has 1) a carbon-carbon double bond between the first and second carbon atom of the longest contiguous chain of carbon atoms, and 2) at least one hydrogen atom bound to the second carbon of the chain. The term “alpha olefin” includes linear and branched alpha olefins and alpha olefins which can have more than one non-aromatic carbon-carbon double bond, unless expressly stated otherwise. In the case of branched olefins, a branch can be at the 2-position of a 1-alkene (a vinylidene) with respect to the olefin double bond. By itself, the term “alpha olefin” does not indicate the presence or absence of heteroatoms and/or the presence or absence of other carbon-carbon double bonds unless explicitly indicated. The terms “hydrocarbon alpha olefin” or “alpha olefin hydrocarbon” refer to alpha olefin compounds containing only hydrogen and carbon.

The term “normal alpha olefin” refers to the general structure H₂C═CRH. The term “vinylidene” refers to the general structure H₂C═CRR′. The term “internal olefin” refers to the general structure RHC═CR′H. Finally, the term “trisubstituted olefin” refers to the general structure RHC═CR′R″. In each case, R, R′, and R″ may be the same or different. The terms “oligomerization” and “oligomerizing” refer to processes which produce an oligomer product comprising at least 20 wt. %, 35 wt. %, 50 wt. %, or 60 wt. % products comprising from 2 to 20 monomer units, including dimers, trimers, tetramers, and so forth. Thus, oligomer refers to a compound that contains from 2 to 20 monomer units, including dimers, trimers, tetramers, and so forth. The terms “oligomerization product” and “oligomer product” include all products made by the “oligomerization” process, including the “oligomers” and products which are not “oligomers” (e.g., products which contain more than 20 monomer units, or solid polymer, as well an unreacted monomer), but exclude other potential non-olefin components of an oligomerization reactor effluent stream, such as catalyst, solvent, and hydrogen, amongst other components. It should be noted that the monomer units in the “oligomer” or “oligomer product” do not have to be the same. For example, these terms are also used generically herein to include olefin homo-oligomers, co-oligomers, and so forth, and thus encompass products derived from any number of different olefin monomers disclosed herein.

A “polyalphaolefin” (PAO) is a mixture of hydrogenated (or alternatively, substantially saturated) oligomers, containing units derived from an alpha olefin monomer. Unless specified otherwise, the PAO can contain units derived from alpha olefin monomer units, which can be the same (hydrogenated or substantially saturated alpha olefin homo-oligomer) or can be different (hydrogenated or substantially saturated alpha olefin co-oligomer). Generally, the alpha olefin monomer utilized to produce the polyalphaolefin can be any alpha olefin monomer described herein (e.g., 1-octene). One having ordinary skill in the art would recognize that the processes for producing the PAO can leave some hydrogenated monomer in the PAO (e.g., less than 1 wt. % based on the total amount of the PAO), and this quantity of hydrogenated monomer can be specified.

For any particular compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to octene (or octenes) includes all linear or branched, acyclic or cyclic, hydrocarbon compounds having eight carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

Features within this disclosure that are provided as minimum values can be alternatively stated as “at least” or “greater than or equal to” any recited minimum value for the feature disclosed herein. Features within this disclosure that are provided as maximum values can be alternatively stated as “less than or equal to” or “below” any recited maximum value for the feature disclosed herein.

Several types of ranges are disclosed herein. When a range of any type is disclosed or claimed, the intent is to disclose or claim individually each possible number that such a range could reasonably encompass, including end points of the range as well as any sub-ranges and combinations of sub-ranges encompassed therein. For example, the weight ratio of the first catalyst composition to the olefin feedstock can be in various ranges. By a disclosure that the weight ratio of the first catalyst composition to the olefin feedstock can range from 1:1 to 1:100, the intent is to recite that the weight ratio can be any ratio within the range and, for example, can include any range or combination of ranges from 1:1 to 1:100, such as from 1:5 to 1:50, from 1:10 to 1:50, or from 1:10 to 1:30, and so forth. Likewise, all other ranges disclosed herein should be interpreted in a manner similar to this example.

In general, an amount, size, formulation, parameter, range, or other quantity or characteristic is “about” or “approximate,” whether or not it is expressly stated to be such. Whether or not modified by the term “about” or “approximately,” the claims include equivalents to the quantities or characteristics.

In the processes disclosed and claimed herein, and not limited thereto, a product in one step can be used as a reactant in another step, or a material can be separated from a reaction mixture. For instance, when a C₁₆ vinylidene dimer is produced in a first step and then utilized in a subsequent step, “all or any portion of” the C₁₆ vinylidene produced in the first step can then be utilized in the subsequent step, even if not specifically stated so. Similarly, for example, when C₃₂ olefins are isolated from a second oligomer product, “all or any portion of” the C₃₂ olefins can be isolated from the second oligomer product, even if not specifically stated so. Likewise, all process steps in the specification and in the claims should be interpreted in a manner similar to these examples.

Although any methods, devices, and materials similar or equivalent to those described herein can be used in the practice or testing of the disclosure, the typical methods, devices, and materials are herein described.

All publications and patents mentioned herein are incorporated by reference in their entirety for the purpose of describing and disclosing, for example, the constructs and methodologies that are described in the publications and patents, which might be used in connection with the present disclosure.

### DETAILED DESCRIPTION

Disclosed herein are processes for oligomerizing 1-octene, first involving the formation of a C₁₆ vinylidene dimer from an olefin feedstock comprising 1-octene, followed by the oligomerization of the C₁₆ vinylidene dimer to produce C₃₂ olefins (1-octene tetramers).

Polyalphaolefins can subsequently be produced by isolating and then hydrogenating the C₃₂ olefins to form the polyalphaolefin product. Polyalphaolefins produced by these processes are encompassed herein. Surprisingly, the oligomerization processes disclosed herein achieve high conversion efficiency in both oligomerization steps and the polyalphaolefins produced demonstrate excellent low-temperature performance and other favorable lubricant characteristics.

### Olefin Oligomerization Processes

An oligomerization process consistent with the present invention can comprise (or consist essentially of, or consist of) (i) contacting a first catalyst composition with an olefin feedstock comprising at least 50 wt. % (or at least 75 wt. %, at least 85 wt. %, at least 95 wt. %, at least 98 wt. %, or at least 99 wt. %) of 1-octene under first oligomerization conditions to produce a first oligomer product comprising a C₁₆ vinylidene dimer, (ii) separating (all or any portion of) the C₁₆ vinylidene dimer from the first oligomer product, and (iii) contacting (all or any portion of) the C₁₆ vinylidene dimer with a second catalyst composition under second oligomerization conditions to produce a second oligomer product comprising at least 50 wt. % (or at least 55 wt. %, at least 60 wt. %, at least 65 wt. %, at least 70 wt. %, or at least 75 wt. %) of C₃₂ olefins.

Generally, the features of the oligomerization processes (e.g., the olefin feedstock, the first and second catalyst compositions, the materials comprising and/or features of the first and second oligomer products, the oligomerization conditions under which the first and second oligomer products are formed, the C₁₆ vinylidene dimer, and the separation step, among others) are independently described herein and these features can be combined in any combination to further describe the disclosed processes. Moreover, additional process steps can be performed before, during, and/or after any of the steps in the processes disclosed herein, and can be utilized without limitation and in any combination to further describe these processes, unless stated otherwise. Further, any oligomer products or polyalphaolefins produced in accordance with the disclosed processes are within the scope of this disclosure and are encompassed herein.

The olefin feedstock in step (i), which comprises at least 50 wt. % of 1-octene, can come from many different sources and have a wide range of compositional attributes. In some aspects, the olefin feedstock can comprise at least 75 wt. % of 1-octene, at least 85 wt. % of 1-octene, at least 95 wt. % of 1-octene, at least 98 wt. % of 1-octene, or at least 99 wt. % of 1-octene. These weight percentages are based on the total weight of olefins in the olefin feedstock.

In addition to the 1-octene, the olefin feedstock can further comprise a minor amount of a C₂ to C₁₄ olefin or a minor amount of any combination of C₂ to C₁₄ olefins. Additionally or alternatively, in addition to the 1-octene, the olefin feedstock can further comprise other C₈ olefins, such as internal C₈ olefins, branched C₈ olefins, and/or vinylidene C₈ olefins in any combination, typically at a total amount ranging from 0.5 to 30 wt. %, and more often, at a total amount ranging from 0.5 to 10 wt. %, from 1 to 10 wt. %, from 1 to 5 wt. %, or from 0.5 to 3 wt. %.

The first catalyst composition in step (i) can comprise any aluminum-based catalyst system suitable for the oligomerization of 1-octene. Thus, the first catalyst composition can comprise an organoaluminum compound. For instance, the organoaluminum compound can comprise, can consist essentially of, or can be, a trialkylaluminum compound. Specific non-limiting examples of suitable compounds can include trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), or combinations thereof. In one aspect, the organoaluminum can comprise, consist essentially of, or can be a trialkylaluminum compound, and the trialkylaluminum compound can comprise, can consist essentially of, or can be, trimethylaluminum (TMA), triethylaluminum (TEA), triisobutylaluminum (TIBA), or any combination thereof, while in another aspect, the organoaluminum compound can comprise, can comprise, consist essentially of, or can be, a trialkylaluminum compound, and the trialkylaluminum compound can comprise, can consist essentially of, or can be, trimethylaluminum (TMA); alternatively, triethylaluminum (TEA); or alternatively, triisobutylaluminum (TIBA).

Alternatively, or additionally, the first catalyst composition in step (i) can comprise any metallocene-based catalyst system suitable for the oligomerization of 1-octene. Thus, the first catalyst composition can comprise a metallocene compound. In an aspect, the metallocene compound can comprise, for example, a transition metal (one or more than one) from Groups 3-10 of the Periodic Table of the Elements. In one aspect, the metallocene compound can comprise a Group 3, 4, 5, or 6 transition metal, or a combination of two or more transition metals. The metallocene compound can comprise chromium, titanium, zirconium, hafnium, vanadium, or a combination thereof, in some aspects, or can comprise chromium, titanium, zirconium, hafnium, or a combination thereof, in other aspects. Accordingly, the metallocene compound can comprise chromium, or titanium, or zirconium, or hafnium, either singly or in combination. In some aspects, the metallocene compound can comprise zirconium. Moreover, catalyst systems comprising two or more metallocene compounds, wherein each metallocene compound independently can comprise chromium, titanium, zirconium, hafnium, vanadium, or a combination thereof, are contemplated and encompassed herein.

The metallocene compound can comprise a bridged metallocene compound. In one aspect, the metallocene compound can comprise a bridged zirconium or hafnium based metallocene compound. In another aspect, the metallocene compound can comprise a bridged zirconium or hafnium based metallocene compound with a carbon bridging atom or a silicon bridging atom. In yet another aspect, the metallocene compound can comprise a bridged zirconium based metallocene with a cyclopentadienyl group and a carbon bridging atom or a silicon bridging atom. In still another aspect, the metallocene compound can comprise a bridged zirconium based metallocene with two cyclopentadienyl groups and a carbon bridging atom or a silicon bridging atom.

In these and other aspects, the bridged metallocene compound can contain an alkyl substituent (e.g., n-butyl, n-propyl) on the bridging atom(s). Additionally or alternatively, the bridged metallocene compound can contain an alkyl substituent, for example, on the bridging atom(s) and/or on a cyclopentadienyl group.

The metallocene compound is not limited solely to the bridged metallocene compounds such as described above. Other suitable bridged metallocene compounds are disclosed in U.S. Pat. Nos. 7,026,494, 7,041,617, 7,226,886, 7,312,283, 7,517,939, and 7,619,047.

In certain aspects of this invention, the first catalyst composition can contain a metallocene compound, and the metallocene compound can comprise an unbridged metallocene compound. In one aspect, the metallocene compound can comprise an unbridged zirconium or hafnium based metallocene compound and/or an unbridged zirconium and/or hafnium based dinuclear metallocene compound. In another aspect, the metallocene compound can comprise an unbridged zirconium or hafnium based metallocene compound containing two cyclopentadienyl groups, two indenyl groups, or a cyclopentadienyl and an indenyl group. In another aspect, the metallocene compound can comprise an unbridged zirconium or hafnium based metallocene compound containing two cyclopentadienyl groups. In another aspect, the metallocene compound can comprise an unbridged zirconium based metallocene compound containing two cyclopentadienyl groups. In another aspect, the metallocene compound can comprise an unbridged zirconium or hafnium based metallocene compound containing two indenyl groups. In another aspect, the metallocene compound can comprise an unbridged zirconium or hafnium based metallocene compound containing a cyclopentadienyl and an indenyl group. In yet another aspect, the metallocene compound can comprise an unbridged zirconium based metallocene compound containing a cyclopentadienyl and an indenyl group. In still another aspect, the metallocene compound can comprise an unbridged zirconium based metallocene compound containing a cyclopentadienyl group and an indenyl group with an alkenyl substituent.

In these and other aspects, the unbridged metallocene compound can contain an alkyl substituent (e.g., n-butyl, n-propyl) on one or both cyclopentadienyl-type groups (e.g., a cyclopentadienyl group, an indenyl group). Accordingly, the metallocene compound can contain an alkyl-substituted cyclopentadienyl group.

Illustrative and non-limiting examples of bridged and unbridged metallocene compounds that are suitable for use as metallocene compounds described herein can include the following compounds (Ph=phenyl):

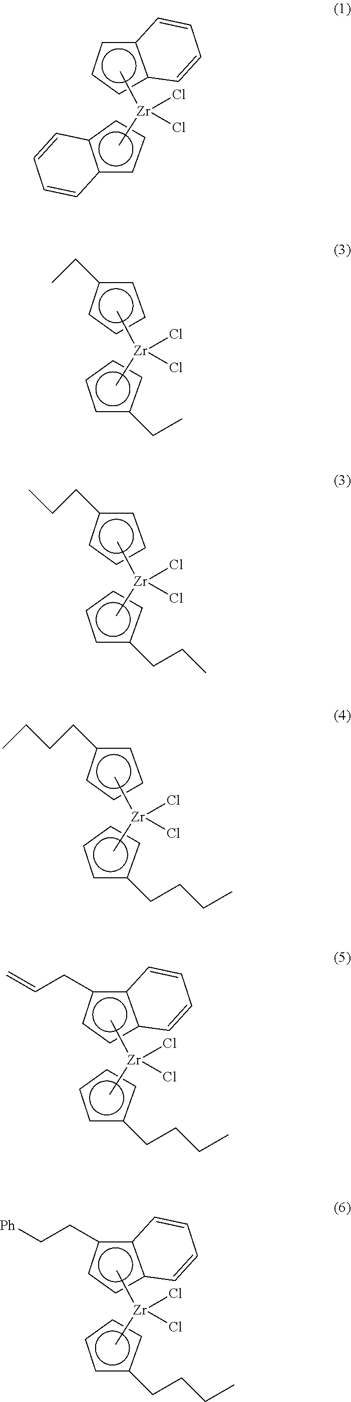

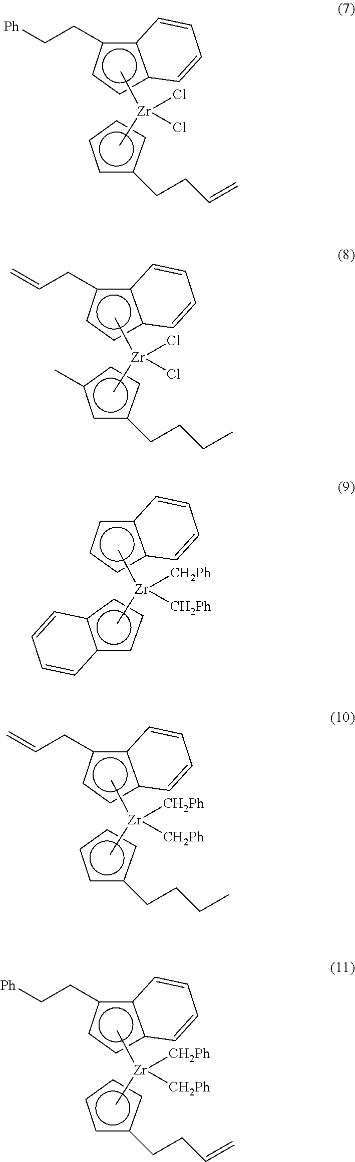

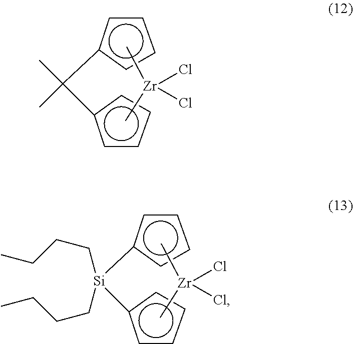

or any combination thereof.

The metallocene compound is not limited solely to bridged and unbridged metallocene compounds such as described above, or to suitable unbridged metallocene compounds disclosed in U.S. Pat. Nos. 7,199,073, 7,226,886, 7,312,283, and 7,619,047. For example, the metallocene compound can comprise an unbridged dinuclear metallocene compound, such as those described in U.S. Pat. Nos. 7,919,639 and 8,080,681. Illustrative and non-limiting examples of dinuclear metallocene compounds suitable for use in the present invention can include the following compounds:

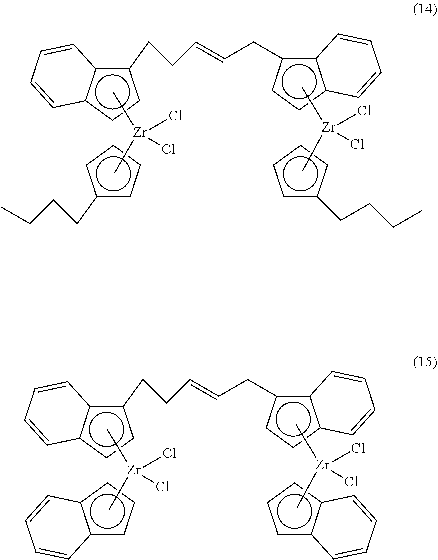

or any combination thereof.

The first catalyst composition in step (i), in addition to a metallocene compound (one or more), can contain a chemically-treated solid oxide. In one aspect, the chemically-treated solid oxide can comprise a solid oxide treated with an electron-withdrawing anion. Alternatively, in another aspect, the chemically-treated solid oxide can comprise a solid oxide treated with an electron-withdrawing anion, the solid oxide containing a Lewis-acidic metal ion. Non-limiting examples of suitable chemically-treated solid oxides are disclosed in, for instance, U.S. Pat. Nos. 7,294,599, 7,601,665, 7,884,163, 8,309,485, 8,623,973, 8,703,886, and 9,023,959.

The solid oxide can encompass oxide materials such as alumina, “mixed oxides” thereof such as silica-alumina, coatings of one oxide on another, and combinations and mixtures thereof. The mixed oxides such as silica-alumina can be single or multiple chemical phases with more than one metal combined with oxygen to form the solid oxide. Examples of mixed oxides that can be used to form a chemically-treated solid oxide, either singly or in combination, can include, but are not limited to, silica-alumina, silica-titania, silica-zirconia, alumina-titania, alumina-zirconia, zinc-aluminate, alumina-boria, silica-boria, aluminophosphate-silica, and titania-zirconia. The solid oxide used herein also can encompass oxide materials such as silica-coated alumina, as described in U.S. Pat. No. 7,884,163.

Accordingly, in one aspect, the solid oxide can comprise silica, alumina, silica-alumina, silica-coated alumina, aluminum phosphate, aluminophosphate, heteropolytungstate, titania, silica-titania, zirconia, silica-zirconia, magnesia, boria, zinc oxide, any mixed oxide thereof, or any combination thereof. In another aspect, the solid oxide can comprise alumina, silica-alumina, silica-coated alumina, aluminum phosphate, aluminophosphate, heteropolytungstate, titania, silica-titania, zirconia, silica-zirconia, magnesia, boria, or zinc oxide, or any combination thereof. In another aspect, the solid oxide can comprise silica, alumina, titania, zirconia, magnesia, boria, zinc oxide, any mixed oxide thereof, or any combination thereof. In yet another aspect, the solid oxide can comprise silica-alumina, silica-coated alumina, silica-titania, silica-zirconia, alumina-boria, or any combination thereof. In still another aspect, the solid oxide can comprise silica, alumina, silica-alumina, silica-coated alumina, or any mixture thereof, alternatively, silica; alternatively, alumina; alternatively, silica-alumina; or alternatively, silica-coated alumina.

The silica-alumina or silica-coated alumina solid oxide materials which can be used can have a silica content from 5 to 95% by weight. In one aspect, the silica content of these solid oxides can be from 10 to 80%, or from 20% to 70%, silica by weight. In another aspect, such materials can have silica contents ranging from 15% to 60%, or from 25% to 50%, silica by weight. The solid oxides contemplated herein can have any suitable surface area, pore volume, and particle size, as would be recognized by those of skill in the art.

The electron-withdrawing component used to treat the solid oxide can be any component that can increase the Lewis or Brönsted acidity of the solid oxide upon treatment (as compared to the solid oxide that is not treated with at least one electron-withdrawing component). According to one aspect, the electron-withdrawing component can be an electron-withdrawing anion derived from a salt, an acid, or other compound, such as a volatile organic compound, that can serve as a source or precursor for that anion. Examples of electron-withdrawing anions can include, but are not limited to, sulfate, bisulfate, fluoride, chloride, bromide, iodide, fluorosulfate, fluoroborate, phosphate, fluorophosphate, trifluoroacetate, triflate, fluorozirconate, fluorotitanate, phospho-tungstate, tungstate, and molybdate, including mixtures and combinations thereof. In addition, other ionic or non-ionic compounds that can serve as sources for these electron-withdrawing anions also can be employed. It is contemplated that the electron-withdrawing anion can be, or can comprise, fluoride, chloride, bromide, phosphate, triflate, bisulfate, or sulfate, or any combination thereof, in some aspects provided herein. In other aspects, the electron-withdrawing anion can comprise sulfate, bisulfate, fluoride, chloride, bromide, iodide, fluorosulfate, fluoroborate, phosphate, fluorophosphate, trifluoroacetate, triflate, fluorozirconate, fluorotitanate, or combinations thereof. Yet, in other aspects, the electron-withdrawing anion can comprise fluoride and/or sulfate.

The chemically-treated solid oxide generally can contain from 1 to 25 wt. % of the electron-withdrawing anion, based on the weight of the chemically-treated solid oxide. In particular aspects provided herein, the chemically-treated solid oxide can contain from 1 to 20 wt. %, from 2 to 20 wt. %, from 3 to 20 wt. %, from 2 to 15 wt. %, from 3 to 15 wt. %, from 3 to 12 wt. %, or from 4 to 10 wt. %, of the electron-withdrawing anion, based on the total weight of the chemically-treated solid oxide.

In an aspect, the chemically-treated solid oxide can comprise fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, fluorided-chlorided silica-coated alumina, sulfated silica-coated alumina, or phosphated silica-coated alumina, or any combination thereof. In another aspect, the chemically-treated solid oxide employed in the catalyst systems described herein can be, or can comprise, a fluorided solid oxide and/or a sulfated solid oxide, non-limiting examples of which can include fluorided alumina, sulfated alumina, fluorided silica-alumina, sulfated silica-alumina, fluorided silica-zirconia, fluorided silica-coated alumina, fluorided-chlorided silica-coated alumina, or sulfated silica-coated alumina, or any combination thereof. In yet another aspect, the chemically-treated solid oxide can comprise fluorided alumina; alternatively, chlorided alumina; alternatively, sulfated alumina; alternatively, fluorided silica-alumina; alternatively, sulfated silica-alumina; alternatively, fluorided silica-zirconia; alternatively, chlorided silica-zirconia; alternatively, sulfated silica-coated alumina; alternatively, fluorided-chlorided silica-coated alumina; or alternatively, fluorided silica-coated alumina. In some aspects, the chemically-treated solid oxide can comprise a fluorided solid oxide, while in other aspects, the chemically-treated solid oxide can comprise a sulfated solid oxide.

Various processes can be used to form chemically-treated solid oxides useful in the present invention. Methods of contacting the solid oxide with the electron-withdrawing component, suitable electron withdrawing components and addition amounts, impregnation with metals or metal ions (e.g., zinc, nickel, vanadium, titanium, silver, copper, gallium, tin, tungsten, molybdenum, zirconium, or combinations thereof), and various calcining procedures and conditions are disclosed in, for example, U.S. Pat. Nos. 6,107,230, 6,165,929, 6,294,494, 6,300,271, 6,316,553, 6,355,594, 6,376,415, 6,388,017, 6,391,816, 6,395,666, 6,524,987, 6,548,441, 6,548,442, 6,576,583, 6,613,712, 6,632,894, 6,667,274, 6,750,302, 7,294,599, 7,601,665, 7,884,163, and 8,309,485. Other suitable processes and procedures for preparing chemically-treated solid oxides (e.g., fluorided solid oxides, sulfated solid oxides, etc.) are well known to those of skill in the art.

While not a requirement, the first catalyst composition in step (i) can further comprise a co-catalyst, and the co-catalyst can comprise an organoaluminum compound, an aluminoxane compound, an organoboron or organoborate compound, an ionizing ionic compound, an organozinc compound, an organomagnesium compound, or an organolithium compound, and this includes any combinations of these materials. Accordingly, the first catalyst composition can further contain, in certain aspects of this invention, an organoaluminum compound; alternatively, an aluminoxane compound; alternatively, an organoboron or organoborate compound; alternatively, an ionizing ionic compound; alternatively, an organozinc compound; alternatively, an organomagnesium compound; or alternatively, an organolithium compound.

These and other suitable co-catalysts that can be used in the first catalyst composition are well known to those of skill in the art including, for example, those disclosed in U.S. Pat. Nos. 3,242,099, 4,794,096, 4,808,561, 5,576,259, 5,807,938, 5,919,983, 7,294,599 7,601,665, 7,884,163, 8,114,946, and 8,309,485.

In some aspects, the first catalyst composition in step (i) can be substantially free of aluminoxanes, organoboron or organoborate compounds, ionizing ionic compounds, and/or other similar materials; alternatively, substantially free of aluminoxanes; alternatively, substantially free or organoboron or organoborate compounds; or alternatively, substantially free of ionizing ionic compounds. In these aspects, the first catalyst composition can have catalyst activity, discussed herein, in the substantial absence of aluminoxanes, organoboron or organoborate compounds, ionizing ionic compounds, and/or other similar materials. For example, a first catalyst composition of the present invention can consist essentially of a metallocene compound, a chemically-treated solid oxide, and an organoaluminum compound, wherein no other materials are present in the catalyst system which would increase/decrease the activity of the catalyst system by more than 10% from the catalyst activity of the catalyst system in the absence of said materials.

When the first catalyst composition comprises a metallocene compound, an organoaluminum compound, and a chemically-treated solid oxide, the organoaluminum compound can comprise, can consist essentially of, or can be, trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum (TNOA), or combinations thereof.

Certain ratios of components may be used to control the first oligomerization reaction in step (i) of the disclosed oligomerization processes. While not being limited thereto, the weight ratio of the first catalyst composition to the olefin feedstock (comprising the 1-octene) in step (i) can fall within any range from 1:1 to 1:100. For instance, the weight ratio of the first catalyst composition to the olefin feedstock can be at least 1:1, 1:5, or 1:10; alternatively, or additionally, the maximum weight ratio of the first catalyst composition to the olefin feedstock can be 1:100, 1:50, or 1:30. Generally, the weight ratio of the first catalyst composition to the olefin feedstock can be in a range from any minimum weight ratio disclosed herein to any maximum weight ratio disclosed herein. Therefore, suitable non-limiting ranges for the weight ratio of the first catalyst composition to the olefin feedstock can include the following ranges: from 1:1 to 1:100, from 1:5 to 1:50, from 1:10 to 1:50, or from 1:10 to 1:30. Other appropriate ranges for the weight ratio of the first catalyst composition to the olefin feedstock are readily apparent from this disclosure.

Generally, the appropriate procedure for contacting (or reacting) the olefin feedstock containing 1-octene and the first catalyst composition in step (i) is not particularly limited. For instance, the olefin feedstock and the first catalyst composition can be contacted in any method or process that produces a first reaction product (the first oligomer product) having an acceptable amount of C₁₆ vinylidene dimers.

The first oligomerization conditions utilized in step (i) and the second oligomerization conditions utilized in step (iii) can comprise any suitable oligomerization temperature. For example, the oligomerization temperature can be in a range from 10° C. to 200° C. in step (i) and (iii). In an aspect, the minimum oligomerization temperature can be 10, 20, 30, 40, 50, 60, or 70° C., alternatively, or additionally, the maximum oligomerization temperature can be 200, 190, 180, 170, 160, 150, or 140° C. Generally, the oligomerization temperature can be in a range from any minimum oligomerization temperature disclosed herein to any maximum oligomerization temperature disclosed herein. Therefore, in some aspects, the oligomerization temperature in step (i) and step (iii), independently, can be in a range from 10° C. to 200° C., from 20° C. to 190° C., from 30° C. to 180° C., from 40° C. to 170° C., from 50° C. to 160° C., from 60° C. to 150° C., or from 70° C. to 140° C. These temperature ranges also are meant to encompass circumstances where step (i) and (iii) are performed at a series of different temperatures, instead of at a single fixed temperature, falling within the respective temperature ranges, wherein at least one temperature is within the recited ranges. Other appropriate oligomerization temperatures and temperature ranges are readily apparent from this disclosure.

In another non-limiting aspect, the first and second oligomerization conditions utilized in step (i) and step (iii) of the oligomerization processes disclosed herein can comprise performing the oligomerization reactions in the presence of hydrogen. The hydrogen partial pressure in the oligomerization reactions can be any pressure of hydrogen that does not adversely affect the oligomerization reactions. While not intending to be bound by theory, hydrogen can be used in the oligomerization processes to control the oligomer distribution. In some non-limiting aspects, the first and second oligomerization conditions can include a partial pressure of hydrogen at least 0.1 psig (6.9 kPa) and often up to and including a partial pressure of 2000 psig (13.8 MPa). For instance, the minimum hydrogen partial pressure can be 1 psig (6.9 kPa), 5 psig (34 kPa), 10 psig (69 kPa), or 25 psig (172 kPa); alternatively, or additionally, the maximum hydrogen partial pressure can be 2000 psig (13.8 MPa), 1500 psig (10.3 MPa), 1000 psig (6.9 MPa), or 500 psig (3.5 MPa). Generally, the first and second oligomer products can be formed, independently, at a hydrogen partial pressure in a range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 5 psig (34 kPa) to 1500 psig (10.3 MPa), from 10 psig (69 kPa) to 1000 psig (6.9 MPa), from 10 psig (69 kPa) to 500 psig (3.5 MPa), or from 25 psig (172 kPa) to 500 psig (3.4 MPa). Other appropriate hydrogen partial pressures are readily apparent from this disclosure.

Any suitable reactor or vessel within an oligomerization reaction system can be used to form the first oligomer product in step (i) and the second oligomer product in step (iii), non-limiting examples of which can include a fixed bed reactor, a stirred tank reactor, a plug flow reactor, and a tubular reactor, including more than one reactor in series or in parallel, and including any combination of reactor types and arrangements. In one aspect, the reaction system can comprise a single reactor (e.g., a single stirred tank reactor), while in another aspect, the reaction system can comprise two reactors in series (or parallel).

In an aspect, the feedstock olefin conversion to the first oligomer product in step (i) can be at least 40 wt. %. The conversion of the feedstock olefin is described as “feedstock olefin conversion” to indicate that the percentage conversion to oligomers, in weight percent, is based on the feedstock olefin and does not include unreacted olefin or non-olefin materials that can be present (e.g., solvent, etc.) in the first oligomer product. The minimum feedstock olefin conversion can be at least 40 wt. %, at least 50 wt. %, at least 60 wt. %, at least 70 wt. %, or at least 75 wt. %, and the maximum conversion can be 95 wt. %, 93 wt. %, 91 wt. %, 89 wt. %, or 88 wt. %. Generally, the feedstock olefin conversion can be in a range from any minimum conversion disclosed herein to any maximum conversion disclosed herein. Non-limiting ranges of feedstock olefin conversion can include, but are not limited to, the following ranges: from 40 to 95 wt. %, from 50 to 93 wt. %, from 60 to 91 wt. %, from 70 to 89 wt. %, or from 75 to 88 wt. %. Other feedstock olefin conversion ranges are readily apparent from this disclosure. These feedstock olefin conversions can be achieved in a batch process or in a flow or continuous process, such as, for example, a single pass through a reactor (e.g., a fixed bed reactor).

The first oligomer product formed in step (i), can be characterized by a relative amount of specific oligomers. For instance, it can be beneficial to maximize the amount of 1-octene dimer (C₁₆), while minimizing heavier oligomers in the first oligomer product. Surprisingly, the oligomerization processes disclosed herein are able to operate at high conversions of the 1-octene monomer to the C₁₆ vinylidene dimer without causing a shift in the resulting oligomer product toward heavier oligomers.

Beneficially, the 1-octene dimer (C₁₆) may be the majority component of the first oligomer product, and the first oligomer product can contain at least 50 wt. %, at least 55 wt. %, at least 60 wt. %, at least 65 wt. %, at least 70 wt. %, or at least 75 wt. % 1-octene dimer (C₁₆), based on total oligomers in the oligomer product, and excluding unreacted olefin monomer.

Specifically, and beneficially, the amount of C₁₆ vinylidene dimer produced by the disclosed oligomerization process is relatively high. Often, vinylidene is desirable for its high reactivity relative to internal and branched dimers of the 1-octene monomer. Also beneficially, this can be accomplished with relatively high conversions of the 1-octene monomer. In certain aspects, the amount of C₁₆ vinylidene dimer in the first oligomer product can be at least 50 wt. 00 at least 60 wt. %, at least 65 wt. %, at least 70 wt. %, at least 75 wt. %, at least 80 wt. %, or at least 85 wt. %, based on total oligomers in the oligomer product, and excluding unreacted olefin monomer. In an aspect, the C₁₆ vinylidene dimer in the first oligomer product comprises 2-hexyl-1-decene. Other isomers may form during vinylidene synthesis due to isomerization, such as, for example, 7-methyl-6-pentadecene and 7-methyl-7-pentadecene.

In addition to the C₁₆ vinylidene dimer, the first oligomer product in step (i) can further comprise unreacted olefin feedstock, tri-substituted dimer, and/or higher oligomers. In an aspect, the oligomerization processes can further comprise a step of separating all or any portion of unreacted olefin feedstock from the first oligomer product using any suitable technique, e.g., wiped film evaporation, distillation, vacuum distillation, short path distillation, filtration, extraction, or any combination thereof. Likewise, the oligomerization processes can further comprise a step of separating all or any portion of the first catalyst composition from the first oligomer product using any suitable technique, e.g., distillation, vacuum distillation, short path distillation, filtration, extraction, wiped film evaporation, or any combination thereof. Optionally, the oligomerization processes can further comprise recycling all or any portion of either or both of the recovered first catalyst composition and the recovered olefin feedstock, for instance, for re-use in step (i) of the oligomerization processes.

Referring now to step (ii) of the oligomerization processes, all or any portion of the C₁₆ vinylidene dimer (e.g., 2-hexyl-1-decene) is separated from the first oligomer product. In an aspect, all or any portion of the C₁₆ vinylidene dimer can be separated from the first reaction product using any suitable technique, e.g., distillation, vacuum distillation, short path distillation, filtration, extraction, wiped film evaporation, or any combination thereof.

The second catalyst composition utilized in step (iii) of the disclosed oligomerization processes can be any catalyst system suitable for the oligomerization of the C₁₆ vinylidene dimer (e.g., 2-hexyl-1-decene). Generally, the second catalyst composition in step (iii) can comprise an aluminum halide compound. The halide of the aluminum halide compound can be bromide, chloride, or iodide; alternatively, bromide; alternatively, chloride; or alternatively, iodide. Examples of suitable aluminum halide compounds include aluminum bromide, aluminum chloride, aluminum iodide, or any combination thereof.

Certain ratios of components may be used to control the second oligomerization reaction in step (iii) of the disclosed oligomerization processes. While not being limited thereto, the weight ratio of the second catalyst composition to the C₁₆ vinylidene dimer (e.g., 2-hexyl-1-decene) in step (iii) can fall within any range from 1:1 to 1:2000. For instance, the weight ratio of the second catalyst composition to the C₁₆ vinylidene dimer can be at least 1:1, 1:5, 1:10, or 1:20; alternatively, or additionally, the maximum weight ratio of the second catalyst composition to the C₁₆ vinylidene dimer can be 1:2000, 1:1000, 1:500, or 1:350. Generally, the weight ratio of the second catalyst composition to the C₁₆ vinylidene dimer can be in a range from any minimum weight ratio disclosed herein to any maximum weight ratio disclosed herein. Therefore, suitable non-limiting ranges for the weight ratio of the second catalyst composition to the C₁₆ vinylidene dimer can include the following ranges: from 1:1 to 1:2000, from 1:5 to 1:1000, from 1:10 to 1:500, or from 1:20 to 1:350. Other appropriate ranges for the weight ratio of the second catalyst composition to the C₁₆ vinylidene dimer are readily apparent from this disclosure.

Generally, the appropriate procedure for contacting (or reacting) the C₁₆ vinylidene dimer (e.g., 2-hexyl-1-decene) and the second catalyst composition in step (iii) is not particularly limited. For instance, the C₁₆ vinylidene dimer and the second catalyst composition can be contacted in any method or process that produces a second reaction product (the second oligomer product) having an acceptable amount of C₃₂ olefins (1-octene tetramers).

In an aspect, the C₁₆ vinylidene dimer (e.g., 2-hexyl-1-decene) conversion to the second oligomer product in step (iii) can be at least 40 wt. %. The conversion of the C₁₆ vinylidene dimer is described as “vinylidene dimer conversion” to indicate that the percentage conversion, in weight percent, is based on the C₁₆ vinylidene dimer and does not include non-olefin materials that can be present (e.g., solvent, etc.). The minimum vinylidene dimer conversion can be at least 40 wt. %, at least 50 wt. %, at least 60 wt. %, at least 70 wt. %, or at least 73 wt. %, and the maximum conversion can be 95 wt. %, 90 wt. %, 87 wt. %, 85 wt. %, or 83 wt. %. Generally, the vinylidene dimer conversion can be in a range from any minimum conversion disclosed herein to any maximum conversion disclosed herein. Non-limiting ranges of vinylidene dimer conversion can include, but are not limited to, the following ranges: from 40 wt. % to 95 wt. %, from 50 wt. % to 90 wt. %, from 60 wt. % to 87 wt. %, from 70 wt. % to 85 wt. %, or from 73 wt. % to 83 wt. %. Other vinylidene dimer conversion ranges are readily apparent from this disclosure. These vinylidene dimer conversions can be achieved in a batch process or in a flow or continuous process, such as, for example, a single pass through a reactor (e.g., a fixed bed reactor).

The second oligomer product formed in step (iii), can be characterized by a relative amount of specific oligomers. For instance, it can be beneficial to maximize the amount of 1-octene tetramers (C₃₂ olefins), while minimizing heavier oligomers in the oligomer product. Surprisingly, the oligomerization processes disclosed herein are able to operate at high conversions of the C₁₆ vinylidene dimer (e.g., 2-hexyl-1-decene) without causing a shift in the resulting oligomer product toward heavier oligomers.

Beneficially, the 1-octene tetramers (C₃₂ olefins) are the majority component of the second oligomer product in step (iii), and the second oligomer product can comprise at least 50 wt. % of C₃₂ olefins, based on total oligomers in the second oligomer product, and excluding unreacted C₁₆ vinylidene or other C₁₆ olefins. In an aspect, the second oligomer product can comprise at least 55 wt. %, at least 60 wt. %, at least 65 wt. %, at least 70 wt. %, or at least 75 wt. % C₃₂ olefins.

In addition to the C₃₂ olefins, the second oligomer product in step (iii) can further comprise less than or equal to 50 wt. % C₄₈ olefins (1-octene hexamers). In an aspect, the second oligomer product in step (iii) can comprise less than or equal to 40 wt. %, less than or equal to 30 wt. %, less than or equal to 25 wt. %, or less than or equal to 20 wt. % C₄₈ olefins (1-octene hexamers).

In the second oligomer product in step (iii), the weight ratio of C₃₂ olefins to the C₄₈ olefins can be from 2:1 to 10:1. For instance, the weight ratio of the C₃₂ olefins to the C₄₈ olefins in the second oligomer product can be at least 2:1, 3:1, or 3.5:1; alternatively, or additionally, the maximum weight ratio of the C₃₂ olefins to the C₄₈ olefins in the second oligomer product can be 10:1, 8:1, 7:1, or 6:1. Generally, the weight ratio of the C₃₂ olefins to the C₄₈ olefins in the second oligomer product can be in a range from any minimum weight ratio disclosed herein to any maximum weight ratio disclosed herein. Therefore, suitable non-limiting ranges for the weight ratio of the C₃₂ olefins to the C₄₈ olefins in the second oligomer product can include the following ranges: from 2:1 to 10:1, from 2:1 to 7:1, from 3:1 to 10:1, from 3:1 to 8:1, or from 3.5:1 to 6:1. Other appropriate ranges for the weight ratio of the C₃₂ olefins to the C₄₈ olefins in the second oligomer product are readily apparent from this disclosure.

In an aspect, the oligomerization processes can further comprise a step of separating all or any portion of the second catalyst composition from the second oligomer product using any suitable technique. For example, wiped film evaporation, distillation, vacuum distillation, short path distillation, filtration, extraction, or any combination thereof can be utilized to separate all or any portion of the second catalyst composition from the second oligomer product. Optionally, the oligomerization processes can further comprise a step of recycling all or any portion of the recovered second catalyst composition, for instance, for re-use in step (iii) of the oligomerization processes.

Aspects of the present invention also are directed to the first oligomer products produced from the olefin feedstock (comprising the 1-octene) and the second oligomer products produced from the C₁₆ vinylidene dimer (e.g., 2-hexyl-1-decene). In an aspect, the present invention can encompass a first oligomer product and/or a second oligomer product produced by any oligomerization processes disclosed herein.

### Processes to Produce a Polyalphaolefin

Processes for producing a polyalphaolefin are also provided herein, and in one aspect, a process to produce a polyalphaolefin can comprise (or consist essentially of, or consist of) (I) contacting a first catalyst composition with an olefin feedstock comprising at least 50 wt. % (or at least 75 wt. %, at least 85 wt. %, at least 95 wt. %, at least 98 wt. %, or at least 99 wt. %) of 1-octene under first oligomerization conditions to produce a first oligomer product comprising a C₁₆ vinylidene dimer, (II) separating (all or any portion of) the C₁₆ vinylidene dimer from the first oligomer product, (III) contacting (all or any portion of) the C₁₆ vinylidene dimer with a second catalyst composition under second oligomerization conditions to produce a second oligomer product comprising at least 50 wt. % (or at least 55 wt. %, at least 60 wt. %, at least 65 wt. %, at least 70 wt. %, or at least 75 wt. %) of C₃₂ olefins, (IV) separating (all or any portion of) the C₃₂ olefins from the second oligomer product, and (V) hydrogenating (all or any portion of) the C₃₂ olefins to form the polyalphaolefin.

Steps (I), (II), and (III) of the disclosed processes to produce a polyalphaolefin are generally the same as described for steps (i), (ii), and (iii), respectively, of the oligomerization processes described hereinabove. Therefore, all descriptions and disclosure of steps (i), (ii), and (iii) of the oligomerization processes described hereinabove apply to steps (I), (II), and (III) of the disclosed processes to produce a polyalphaolefin, respectively.

Generally, the features of step (IV) and step (V) of the processes to produce a polyalphaolefin (e.g., the separation step and the hydrogenation temperatures and other conditions under which the polyalphaolefin products are formed, among others) are independently described herein and these features can be combined in any combination to further describe the disclosed processes. Moreover, additional process steps can be performed before, during, and/or after any of the steps in the processes to produce a polyalphaolefin disclosed herein, and can be utilized without limitation and in any combination to further describe these processes, unless stated otherwise. Further, any oligomer products or polyalphaolefins produced in accordance with the disclosed processes are within the scope of this disclosure and are encompassed herein.

In step (IV), all or any portion of the C₃₂ olefins (1-octene tetramers) are separated from the second oligomer product. In an aspect, all or any portion of the C₃₂ olefins can be separated from the second oligomer product using any suitable technique, e.g., distillation, vacuum distillation, short path distillation, filtration, extraction, wiped film evaporation, or any combination thereof.

Referring now to step (V), all or any portion of the C₃₂ olefins (1-octene tetramers) are hydrogenated to form the polyalphaolefin. In an aspect, hydrogenating all or any portion of the C₃₂ olefins can utilize any suitable metal catalyst including, for example, platinum, rhenium, palladium, nickel, or any combination thereof.

The hydrogenation in step (V) can be performed at any suitable temperature. For example, the hydrogenation temperature can be in a range from 10° C. to 200° C. In an aspect, the minimum hydrogenation temperature can be 10, 20, 50, or 80° C., alternatively, or additionally, the maximum hydrogenation temperature can be 200, 180, 160, or 140° C. Generally, the hydrogenation temperature can be in a range from any minimum hydrogenation temperature disclosed herein to any maximum hydrogenation temperature disclosed herein. Therefore, in some aspects, the oligomerization temperature can be in a range from 10° C. to 200° C., from 20° C. to 180° C., from 50° C. to 160° C., or from 80° C. to 140° C. These temperature ranges also are meant to encompass circumstances where step (V) is performed at a series of different temperatures, instead of at a single fixed temperature, falling within the respective temperature ranges, wherein at least one temperature is within the recited ranges. Other appropriate hydrogenation temperatures and temperature ranges are readily apparent from this disclosure.

Moreover, this invention also encompasses the polyalphaolefins produced by the processes described hereinabove, and further, having any one or more of the compositional and/or viscosity properties described herein.

The polyalphaolefins produced by the processes provided herein can comprise at least 70 wt. % C₃₂ alkanes (hydrogenated 1-octene tetramers). In an aspect, the polyalphaolefins can comprise at least 72 wt. %, at least 75 wt. %, at least 77 wt. %, at least 78 wt. %, or at least 80 wt. % C₃₂ alkanes. While not limited thereto, the C₃₂ alkanes can comprise at least 25 wt. %, at least 35 wt. %, at least 40 wt. %, at least 45 wt. % at least 50 wt. % or at least 60 wt. % of a compound having the structure:

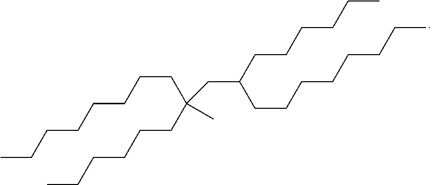 [SMILES: CCCCCCCCC(CCCCCC)CC(C)(CCCCCC)CCCCCCCC]

Advantageously, the polyalphaolefins produced by the processes provided herein can have a 100° C. kinematic viscosity (KV100) in a range from 3.5 to 5 cSt, or a pour point in a range from −90 to −50° C., or a flash point in a range from 200 to 300° C., or a viscosity index in a range from 90 to 120, or any combination of two or more of these properties.

### Polyalphaolefin Compositions

This invention is also directed to, and encompasses, the polyalphaolefins produced using the processes disclosed herein (polyalphaolefins also may be referred to as PAOs or polyalphaolefin compositions). The polyalphaolefins produced can be characterized by various analytical techniques known and used in the polyalphaolefin and lubricant industry. Additionally, lubricant compositions can be formed from, and/or can comprise, the polyalphaolefins of this invention, whose typical properties are provided below.

An illustrative and non-limiting example of a first polyalphaolefin, produced using any of the processes disclosed herein, can have a 100° C. kinematic viscosity (KV100) in a range from 3.5 to 5 cSt, a pour point in a range from −90 to −50° C., and a viscosity index in a range from 90 to 120. While not required, the first polyalphaolefin often can comprise at least 70 wt. %, and more often, at least 72 wt. %, at least 75 wt. %, at least 77 wt. %, at least 78 wt. %, or at least 80 wt. %, of C₃₂ alkanes (hydrogenated 1-octene tetramers).

An illustrative and non-limiting example of a second polyalphaolefin provided herein can comprise at least 70 wt. % (or at least 72 wt. %, at least 75 wt. %, at least 77 wt. %, at least 78 wt. %, or at least 80 wt. %) of C₃₂ alkanes (hydrogenated 1-octene tetramers) and have a 100° C. kinematic viscosity (KV100) in a range from 3.5 to 5 cSt, a pour point in a range from −90 to −50° C., and a flash point in a range from 200 to 300° C.

An illustrative and non-limiting example of a third polyalphaolefin provided herein can comprise at least 70 wt. % (or at least 72 wt. %, at least 75 wt. %, at least 77 wt. %, at least 78 wt. %, or at least 80 wt. %) of C₃₂ alkanes (hydrogenated 1-octene tetramers) and can have a 100° C. kinematic viscosity (KV100) in a range from 3.5 to 5 cSt, a pour point in a range from −90 to −50° C., and a viscosity index in a range from 90 to 120.

An illustrative and non-limiting example of a fourth polyalphaolefin provided herein can comprise at least 70 wt. % (or at least 72 wt. %, at least 75 wt. %, at least 77 wt. %, at least 78 wt. %, or at least 80 wt. %) of C₃₂ alkanes (hydrogenated 1-octene tetramers) and have a 100° C. kinematic viscosity in a range from 3.5 to 5 cSt. For the fourth polyalphaolefin, the C₃₂ alkanes comprise at least 25 wt. % (or at least 35 wt. %, at least 40 wt. % at least 45 wt. % at least 50 wt. %, or at least 60 wt. %) of a compound having the structure:

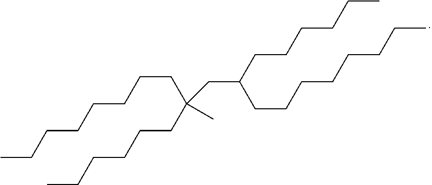 [SMILES: CCCCCCCCC(CCCCCC)CC(C)(CCCCCC)CCCCCCCC]

Further, these illustrative first, second, third, and fourth polyalphaolefins consistent with the present invention also can have any of the properties listed below and in any combination, unless indicated otherwise.

The 100° C. kinematic viscosity (KV100) of the polyalphaolefins often can range from 3.7 to 4.8 cSt. For instance, the polyalphaolefins can have a minimum KV100 of 3.7, 3.9, 4, or 4.2 cSt; additionally or alternatively, the maximum KV100 of the polyalphaolefins can be 4.8, 4.6, or 4.5. Generally, the 100° C. kinematic viscosity of the polyalphaolefins can be in a range from any minimum KV100 disclosed herein to any maximum KV100 disclosed herein. Therefore, suitable non-limiting ranges for the 100° C. kinematic viscosity of the polyalphaolefins can include the following ranges: from 3.7 to 4.8 cSt, from 3.7 to 4.6 cSt, from 3.9 to 4.6 cSt, from 4 to 4.5 cSt, from 4.2 to 4.8 cSt, or from 4.2 to 4.6 cSt. Beneficially, the high 100° C. kinematic viscosity indicates that the polyalphaolefins remain relatively thick and maintain a high viscosity at elevated temperatures. The 100° C. kinematic viscosity is determined in accordance with ASTM D7042-04.

The pour point of the polyalphaolefins often can range from −90° C. to −50° C. For instance, the polyalphaolefins can have a minimum pour point of −90, −85, or −80° C.; additionally or alternatively, the maximum pour point of the polyalphaolefins can be −50, −60, or −70° C. Generally, the pour point of the polyalphaolefins can be in a range from any minimum pour point disclosed herein to any maximum pour point disclosed herein. Therefore, suitable non-limiting ranges for the pour point of the polyalphaolefins can include the following ranges: from −90° C. to −50° C., from −85° C. to −65° C., from −85° C. to −70° C., or from −80° C. to −70° C. Advantageously, the low pour point indicates that the polyalphaolefins can function in cold temperatures without solidifying or becoming overly viscous. The pour point is determined in accordance with ASTM D5950.

The viscosity index of the polyalphaolefins often can range from 90 to 120. For instance, the polyalphaolefins can have a minimum viscosity index of 90, 95, 100, or 105; additionally or alternatively, the maximum viscosity index of the polyalphaolefins can be 120, 115, or 110. Generally, the viscosity index of the polyalphaolefins can be in a range from any minimum viscosity index disclosed herein to any maximum viscosity index disclosed herein. Therefore, suitable non-limiting ranges for the viscosity index of the polyalphaolefins can include the following ranges: from 90 to 120, from 95 to 115, from 100 to 115, or from 105 to 110.

The polyalphaolefins often can comprise at least 70 wt. % of C₃₂ alkanes (hydrogenated 1-octene tetramers). More often, the polyalphaolefins can comprise at least 72 wt. %, at least 75 wt. %, at least 77 wt. %, at least 78 wt. %, or at least 80 wt. % of C₃₂ alkanes. The amount of C₃₂ alkanes in the (first, second, third, and fourth) polyalphaolefins can be determined using the GC analytical procedure described in the Example section below. Additionally or alternatively, the polyalphaolefins or the C₃₂ alkanes can comprise at least 25 wt. %, at least 35 wt. %, at least 40 wt. %, at least 45 wt. %, at least 50 wt. %, or at least 60 wt. % of a compound having the structure:

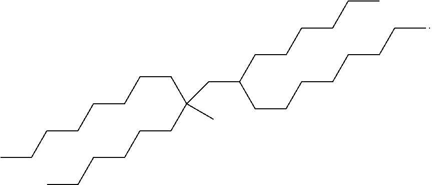 [SMILES: CCCCCCCCC(CCCCCC)CC(C)(CCCCCC)CCCCCCCC]

Importantly, the compound having the structure shown above is believed to be the main C₃₂ alkane isomer present in the polyalphaolefins. While not wishing to be bound by theory, the substitution pattern on this major isomer may confer the superior low temperature viscosity and other beneficial properties to the polyalphaolefins.

The flash point of the polyalphaolefins often can range from 200° C. to 300° C. For instance, the polyalphaolefins can have a minimum flash point of 200, 210, 215, 220, 225, 230, or 235° C.; additionally or alternatively, the maximum flash point of the polyalphaolefins can be 300, 290, 280, 270, 260, 250, or 240° C. Generally, the flash point of the polyalphaolefins can be in a range from any minimum flash point disclosed herein to any maximum flash point disclosed herein. Therefore, suitable non-limiting ranges for the flash point of the polyalphaolefins can include the following ranges: from 200° C. to 300° C., from 210° C. to 300° C., from 215° C. to 290° C., from 220° C. to 280° C., from 220° C. to 260° C., from 225° C. to 270° C., from 230° C. to 270° C., from 230° C. to 260° C., from 230° C. to 250° C., or from 235° C. to 240° C. Beneficially, the high flash point indicates that the polyalphaolefins can be thermally stable at elevated temperatures. The flash point is determined in accordance with ASTM D92.

The 40° C. kinematic viscosity (KV40) of the polyalphaolefins can often range from 5 to 50 cSt. For instance, the polyalphaolefins can have a minimum KV40 of 5, 10, 15, or 20 cSt; additionally or alternatively, the maximum KV40 of the polyalphaolefins can be 50, 40, 30, or 25 cSt. Generally, the 40° C. kinematic viscosity of the polyalphaolefins can be in a range from any minimum KV40 disclosed herein to any maximum KV40 disclosed herein. Therefore, suitable non-limiting ranges for the 40° C. kinematic viscosity of the polyalphaolefins can include the following ranges: from 5 to 50 cSt, from 10 to 40 cSt, from 15 to 30 cSt, or from 20 to 25 cSt. KV40 is determined in accordance with ASTM D7042-04.

The −40° C. kinematic viscosity (KV-40) of the polyalphaolefins can often range from 2500 to 4500 cSt. For instance, the polyalphaolefins can have a minimum KV-40 of 2500, 3000, 3300, 3600, or 3700 cSt; additionally or alternatively, the maximum KV-40 of the polyalphaolefins can be 4500, 4300, 4000, or 3800 cSt. Generally, the −40° C. kinematic viscosity of the polyalphaolefins can be in a range from any minimum KV-40 disclosed herein to any maximum KV-40 disclosed herein. Therefore, suitable non-limiting ranges for the −40° C. kinematic viscosity of the polyalphaolefins can include the following ranges: from 2500 to 4500 cSt, from 3000 to 4500 cSt, from 3300 to 4300 cSt, from 3600 to 4000 cSt, or from 3700 to 3800 cSt. KV-40 is determined in accordance with ASTM D7042-04.

The Noack volatility (as measured by ASTM D5800) of the polyalphaolefins can often range from 5% to 20%. For instance, the polyalphaolefins can have a minimum Noack volatility of 5%, 7%, 8%, 10%, or 11%; additionally or alternatively, the maximum Noack volatility of the polyalphaolefins can be 20%, 15%, 14%, or 13%. Generally, the Noack volatility of the polyalphaolefins can be in a range from any minimum Noack volatility disclosed herein to any maximum Noack volatility disclosed herein. Therefore, suitable non-limiting ranges for the Noack volatility of the polyalphaolefins can include the following ranges: from 5% to 20%, from 7% to 15%, from 8% to 15%, from 10% to 14%, or from 11% to 13%.

Lubricant compositions can be formed from, and/or can comprise, the polyalphaolefins produced by the processes of the present invention. Consistent with an aspect of the present invention, a lubricant composition can comprise (or consist essentially of, or consist of) a polyalphaolefin described herein with or without additives, such as an anti-wear additive, a dispersant, a viscosity modifier, a friction modifier/reducer, a detergent, a demulsifier, a defoamant, an antioxidant, an extreme pressure agent, a rust/corrosion inhibitor, a metal passivator, a pour point depressant, a thickener, and the like, or any combination thereof. Typical lubricant compositions and additives are disclosed in, for example, U.S. Patent Publication No. 2010/0317904.

Viscosity index improvers (also known as viscosity modifiers and viscosity improvers) can provide lubricant compositions with high and low temperature operability. These additives can impart shear stability at elevated temperatures and acceptable viscosity at low temperatures. Suitable viscosity index improvers can include high molecular weight hydrocarbons, olefin polymers and copolymers, polyesters, and viscosity index improver dispersants that function as both a viscosity index improver and a dispersant. Viscosity index improvers can have molecular weights ranging from 10,000 Da to 1,000,000 Da, from 20,000 Da to 500,000 Da, or from 50,000 Da to 200,000 Da.

Viscosity index improvers can include polymers and copolymers of methacrylate, butadiene, olefins, or alkylated styrenes. Exemplary viscosity index improvers include, but are not limited to, polyisobutylene, copolymers of ethylene and propylene, hydrogenated block copolymers of styrene and isoprene, polyacrylates (e.g., polymers and/or copolymers of various chain length acrylates), and polymethacrylates (e.g., polymers and/or copolymers of various chain length alkyl methacrylates).

### EXAMPLES

The disclosure is further illustrated by the following examples, which are not to be construed in any way as imposing limitations to the scope of this disclosure. Various other aspects, modifications, and equivalents thereof, which after reading the description herein, can suggest themselves to one of ordinary skill in the art without departing from the spirit of the present disclosure or the scope of the appended claims.

Gas chromatographic (GC) analyses were performed using an on-column injection method on an Agilent 7890B instrument equipped with an Agilent CP-SimDist UltiMetal column equipped with a flame ionization detector (FID). Initial oven temperature was 40° C. for 7 minutes and was increased 20° C./min to 430° C. and held for 20 minutes. Hydrogen was used as a carrier gas and nitrogen was used as a makeup gas. Data analysis was performed using CompassCDS software.

¹³C NMR analyses were performed on a Bruker Avance II 400 MHz NMR. The analyses were performed using C₆D₆ as a solvent. The ¹³C NMR analyses were performed at a field strength of approximately 100.6 MHz, a power level of approximately 87.07 W, a P1 of 13.65 psec, and a PL1 of −4.00 dB.

¹H NMR analyses were performed on a Bruker Avance II 400 MHz NMR. Spectra were recorded in C₆D₆ and are reported relative to SiMe₄ as determined by reference to the residual ¹H solvent peak. Integration of the following chemical shift ranges were used to determine the relative amounts of olefin end group: Vinylidene: 4.55-4.75 ppm, Trisubstituted: 4.95-5.15 ppm, Internal: 5.20-5.45 ppm.

Kinematic viscosities at 100° C., 40° C., and −40° C. were determined using an Anton Paar Automatic Kinematic Viscometer SVM 3001 in accordance with ASTM D7042-04 (Stabinger viscometer method) at the respective temperatures, and the results are reported in centistokes (cSt). Pour point is a measurement of the temperature at which the sample will begin to flow under carefully controlled conditions. Pour point was determined using a CPP 5Gs Automated Cloud and Pour Point Analyzer in accordance with ASTM D5950 (automatic tilt method), and the results are reported in ° C. The flash point was determined using a PAC Herzog OptiFlash Cleveland Open Cup instrument in accordance with ASTM D92 (but with an electric ignition source instead of a gas flame), and the results are reported in ° C. Noack volatility was determined in accordance with ASTM D5800. Viscosity index was determined in accordance with ASTM D2270.

### Examples 1-8

Oligomerization of 1-Octene with an Organoaluminum Catalyst

First, 1-octene was oligomerized using an organoaluminum compound. Approximately 2-L of anhydrous 1-octene (Chevron Philips Chemical Company) were charged under nitrogen atmosphere to a 3-L round bottom flask equipped with a heating unit, condenser, and magnetic stirrer. The temperature was increased to 40° C. while stirring. Then, 50 grams of triisobutylaluminum (TIBA) were added to the flask. The dimerization reaction was conducted for about 18 hours at 116° C. After cooling to room temperature, the solution was analyzed by GC-FID. The data indicated that the reaction was incomplete, thus an additional 50 grams of TIBA were added to the flask and the dimerization reaction was conducted for about 24 hours at 116° C. to yield an olefin feedstock conversion of 87 wt. %. The solution was then cooled to room temperature and quenched over several hours with 250 mL of 5% aqueous KOH under nitrogen purge. The organic fraction was isolated using a separatory funnel and washed three times with de-ionized water to yield a colorless, clean C₁₆ fraction. The majority of the C₁₆ material was composed of the vinylidene dimer, 2-hexyl-1-decene (77 wt. %), but a noticeable amount (14 wt. %) isomerized to the tri-substituted olefin. Small amounts of other isomers and paraffins were observed. The 2-hexyl-1-decene vinylidene material was isolated overhead by vacuum distillation.

Oligomerization of 2-Hexyl-1-Decene with Various Catalysts

Tetramers of 1-octene were produced by contacting the isolated 2-hexyl-1-decene vinylidene material and an acid catalyst under oligomerization conditions. The 2-hexyl-1-decene was stirred and heated to the target reaction temperature in a 20 mL glass vial in a dry box and the solid acid catalyst was added in one slug along with 40 ppm of adventitious H₂O co-catalyst. As shown in Table I, aluminum bromide (AlBr₃) was utilized as the catalyst at various loadings for Examples 1-5 and 7, aluminum chloride (AlCl₃) was utilized as the catalyst for Example 6, and iron (III) chloride (FeCl₃) was utilized as the catalyst for Example 8. The solution was held at the designated reaction temperature for the reaction time in Table I.

As shown in Table I, Example 1 utilized a relatively high AlBr₃ catalyst loading of 7 wt. % and was heated at 100° C. for 2 hours, resulting in an excellent 1-octene dimer (C₁₆) to 1-octene tetramer (C₃₂) conversion of 80 wt. %. Additionally, high C₃₂ selectivity was observed with a C₃₂ to C₄₈ weight ratio of 4.0. The reaction did not proceed further when allowed to react for an additional 2 hours which was consistent with complete catalyst deactivation. Although the AlBr₃ catalyst loading in Examples 2-5 was reduced, the conversion unexpectedly did not drop significantly, indicating that saturation kinetics had been achieved. Example 6 utilized 3 wt. % AlCl₃ catalyst and demonstrated an excellent 1-octene dimer (C₁₆) to 1-octene tetramer (C₃₂) conversion of 80 wt. % and a high C₃₂ selectivity was observed with a C₃₂ to C₄₈ weight ratio of 5.2. Example 7 utilized only 0.01 wt. % AlBr₃ catalyst and demonstrated a low conversion of 1-octene dimer (C₁₆) to 1-octene tetramer (C₃₂) of only 1.5 wt. %. Similarly, Example 8 utilized FeCl₃ catalyst and demonstrated a low conversion of 1-octene dimer (C₁₆) to 1-octene tetramer (C₃₂) of 10.6 wt. %.

### Examples 9-10 and 20 and Comparative Examples C11-C13

Large Scale Oligomerization of 2-Hexyl-1-Decene with AlBr₃ Catalyst

Approximately 535 grams of the 2-hexyl-1-decene (vinylidene dimer, C₁₆) were charged under nitrogen atmosphere to a 1-L round bottom flask equipped with a heating unit, condenser, and magnetic stirrer. The temperature was increased to 85° C. while stirring. Then, 5 grams of the AlBr₃ catalyst were added to the flask. Temperature monitoring showed a rapid increase in temperature to 140° C., which held for about 5 minutes. Then, an additional 10 grams of AlBr₃ were added to the flask and no further temperature increase was observed. The resulting orange-colored solution was kept above 100° C. for 1 hour and then was cooled to 50° C. over 30 minutes. After cooling, the solution was quenched with 10 wt. % aqueous NaOH and washed twice with de-ionized water. The solution was then dried over MgSO₄ yielding a slightly viscous pale-yellow liquid. The liquid was vacuum distilled (2 torr, 230° C.) to remove the light C₁₆ reactant overhead. The C₃₂ (and higher oligomer) fraction from the bottoms was collected and was 390 g of colorless liquid.

The C₃₂ (and higher oligomer) fraction from the bottoms was characterized further by gas chromatography (GC) to gain a better understanding of the composition. GC analysis indicated isolation of a high purity C₃₂ material (greater than 97 wt. %) with barely resolvable higher molecular weight fractions.

The C₃₂ fraction from the bottoms was hydrogenated as follows. Approximately 15.6 g of the C₃₂ fraction, 1 g of a palladium on carbon catalyst, and 50 mL of tridecane were charged under nitrogen atmosphere to a 250 mL round bottom flask. The mixture was purged with nitrogen gas and then a flow stream of hydrogen was bubbled continuously through the mixture. The temperature of the slurry was increased to 120° C. and maintained for 1 week to complete the hydrogenation reaction. After the reactor contents were cooled to ambient temperature and residual catalyst was removed by filtration, the resultant hydrogenated C₃₂ fraction was a water white liquid.

To minimize cracking and degradation, the bottoms containing the hydrogenated C₃₂ product was collected in three batches. The 100° C. kinematic viscosity (KV100), 40° C. kinematic viscosity (KV40), and viscosity index (VI) of the three hydrogenated bottoms batches (Batches 1-3) were measured and are summarized in Table II. As shown in Table II, the physical properties varied significantly between the three batches.

It is believed that the source of the viscosity variation between the three bottoms batches was caused by small amounts of heavier oligomers insufficiently resolved by GC analysis. Thus, the unhydrogenated C₃₂ fraction from the bottoms was distilled overhead to separate it from the heavier co-products. The KV100 of the isolated unhydrogenated C₃₂ fraction (Example 9) was 4.21 cSt, the KV40 was 20.25 cSt, and the VI was 112. Additionally, the KV-40 was 3623 cSt, which is outstanding for a C₈ based material.

### Hydrogenation of Isolated C₃₂ Oligomer Product

The isolated C₃₂ fraction was hydrogenated as follows. Approximately 15.6 g of the isolated C₃₂ product, 1 g of a palladium on carbon catalyst, and 50 mL of tridecane were charged under nitrogen atmosphere to a 250 mL round bottom flask. The mixture was purged with nitrogen gas and then a flow stream of hydrogen was bubbled continuously through the mixture. The temperature of the slurry was increased to 120° C. and maintained for 1 week to complete the hydrogenation reaction. After the reactor contents were cooled to ambient temperature and residual catalyst was removed by filtration, the resultant hydrogenated C₃₂ product was a water white liquid.

Various standard physical properties were measured for the hydrogenated C₃₂ overhead product of Example 10 (polyalphaolefin, PAO) and are summarized in Table III. Two commercially available low viscosity products are shown in Table III for comparison: a gas-to-liquids Group III+ material (Comparative Example 11) and a Korean refinery Group III mineral oil (Comparative Example 12). Additionally, a PAO 4 material (Comparative Example C13) is shown in Table III for comparison and comprises mostly C₃₀ alkanes (around 80 wt. %).

Unexpectedly, the hydrogenated C₃₂ PAO product (Example 10) possessed several unique features which qualify it as a valuable lubricant base oil. Example 10 had excellent low temperature properties similar to C₁₀-based PAOs, while maintaining low volatility. This lower volatility can lead to better lubricant stability over extended operation at high temperatures, while start-up and low temperature operations can be superior to gas-to-liquid and refinery base stocks. Further, as shown in Table III, Example 10 had a surprising combination of the highest KV100 and highest flash point, and the lowest viscosity index and lowest pour point, as compared to Examples C11-C13.

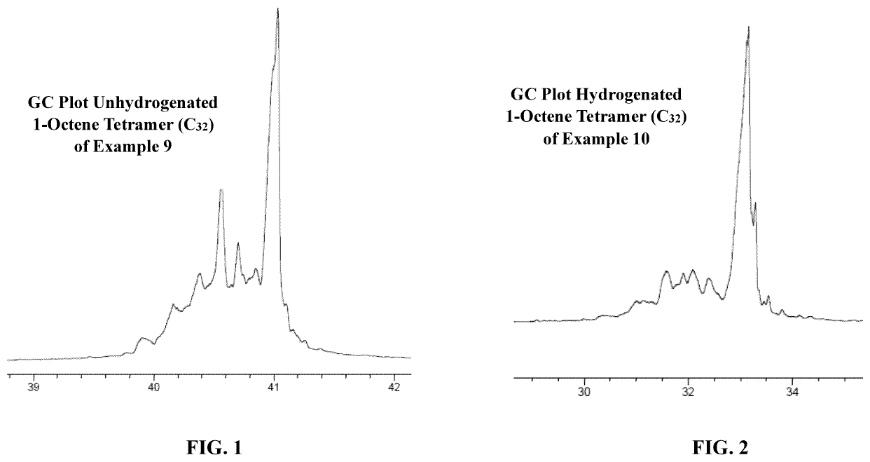

The hydrogenated C₃₂ PAO product was further characterized by gas chromatography (GC), ¹H-NMR spectroscopy, and ¹³C-NMR spectroscopy. FIG. 1 provides a portion of the GC plot of the unhydrogenated C₃₂ product of Example 9 and FIG. 2 provides a portion of the GC plot of the hydrogenated C₃₂ PAO product of Example 10. Compared to FIG. 1, FIG. 2 shows a reduction in the total number of peaks which is consistent with multiple olefinic isomers being hydrogenated to a single hydrocarbon. These GC plots show that one major product dominates in both unhydrogenated and hydrogenated C₃₂ products which will be discussed in more detail below.

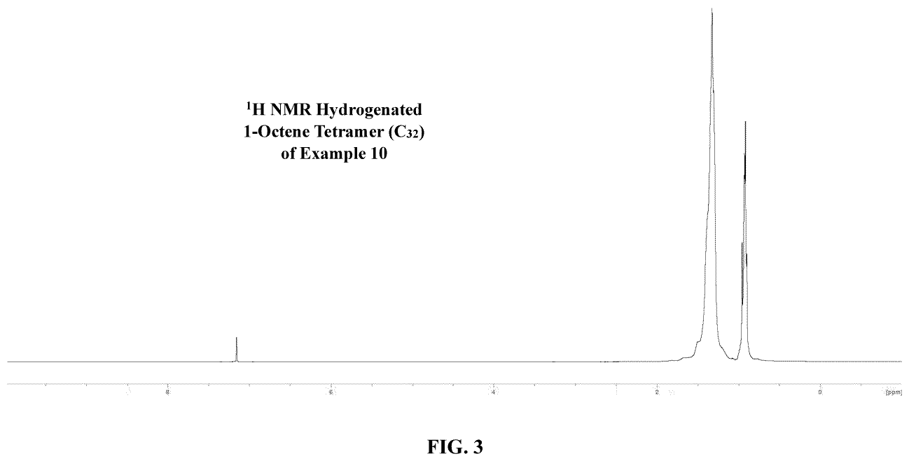

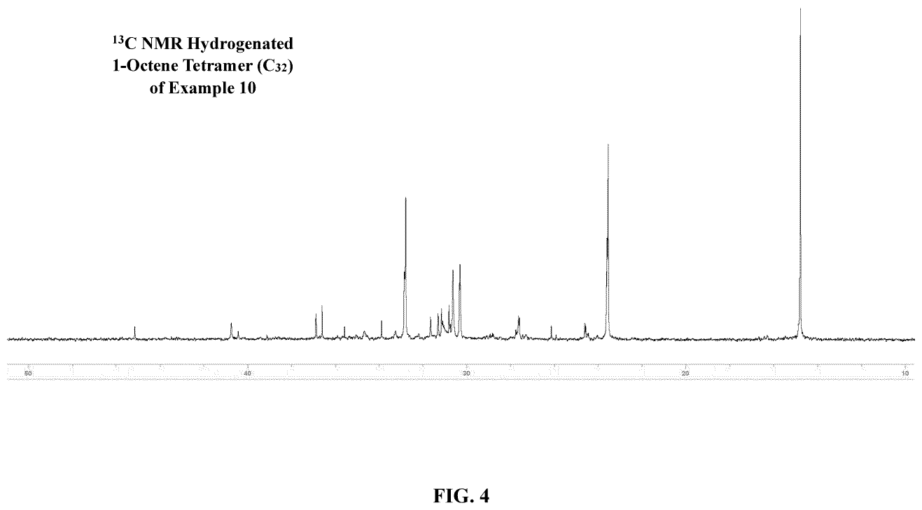

FIG. 3 provides the ¹H-NMR spectra of the hydrogenated C₃₂ PAO product of Example 10. The ¹H-NMR spectra confirmed the complete hydrogenation of C═C bonds, showing no signal in the olefinic region. FIG. 4 provides the ¹³C-NMR spectra of the hydrogenated C₃₂ PAO product of Example 10. The ¹³C-NMR spectra shows a limited number of peaks (at ~30). DEPT-NMR confirmed two quaternary carbon peaks (δ 36.85, 35.55), a chain-end methyl (δ 14.76) peak, and methine peaks. Overall, the analytical data demonstrates that the main molecular structure present (at approximately 40 wt. %) in the hydrogenated C₃₂ PAO product was:

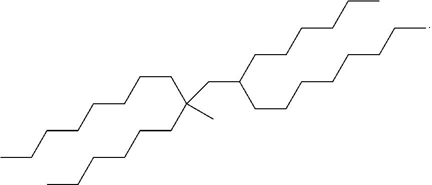 [SMILES: CCCCCCCCC(CCCCCC)CC(C)(CCCCCC)CCCCCCCC]

As shown in the chemical structure depicted above, this C₃₂ hydrocarbon contains a quaternary carbon connected to a methyl group and three long chain alkyl groups, one of which is branched via a tertiary carbon. While not wishing to be bound by theory, the substitution pattern on this major isomer may confer the superior low temperature viscosity properties and other beneficial properties of the PAO of Example 10, as summarized in Table III.

Example 20 was performed in a manner similar to that of Example 10, and its properties are summarized in Table III. Similar to Example 10, and unexpectedly, the hydrogenated C₃₂ PAO product of Example 20 possessed a combination of unique features for a lubricant base oil. As shown in Table III, Example 20 had a surprising combination of the highest KV100 and highest flash point, and the lowest viscosity index and lowest pour point, as compared to Examples C11-C13.

### Examples 14-19

### Investigation of Viscosity Modifiers

Efforts to increase the viscosity index of the hydrogenated C₃₂ PAO product of Example 10 were also explored. Commercial high viscosity metallocene polyalphaolefins (mPAOs) including mPAO 65 (Example 14) and mPAO 150 (Example 15) (both Chevron Phillips Chemical Company) were evaluated as viscosity index enhancers. Both mPAO 65 (Example 14) and mPAO 150 (Example 15) increased the viscosity index of the hydrogenated C₃₂ PAO product significantly, as shown in Table IV. Additionally, both mPAO 65 (Example 14) and mPAO 150 (Example 15) increased the 100° C. kinematic viscosity (KV100). Specifically, the addition of 5 wt. % mPAO 150 (Example 15) to the hydrogenated C₃₂ PAO product (Example 10) created a base oil with a viscosity index of 125 and KV100 of 5.12 cSt. Thus, mPAOs (Examples 14-15) were found to be effective additives to increase the viscosity index and KV100 of the hydrogenated C₃₂ PAO product (Example 10) as needed.

Additionally, styrenic thermoplastic elastomers additives were explored as potential viscosity modifiers. These polymer additives demonstrate favorable compatibility with mineral oils, which share many properties with the hydrogenated C₃₂PAO product of Example 10. Thus, samples of two hydrogenated styrenic block copolymers were obtained (Kuraray) for evaluation: Septon™ 1020 and Septon™ 2002 (see Table V). Septon™ 1020 is a di-block copolymer (styrene-ethylenepropylene or SEP) with 36% styrene content and is in powder form. Septon™ 2002 is a tri-block copolymer (styrene-ethylenepropylene-styrene or SEPS) having 30% styrene content and is in pellet form. Both polymers dissolved slowly in the hydrogenated C₃₂ PAO and required heating and stirring for multiple hours. Maximum solubility was lower than 1 wt. % for both additives. As shown in Table V, 0.2 wt. % of Septon™ 1020 was utilized for Example 16, 0.3 wt. % of Septon™ 1020 was utilized for Example 17, 0.2 wt. % of Septon™ 2002 was utilized for Example 18, and 0.5 wt. % of Septon™ 2002 was utilized for Example 19. Example 16 demonstrates that only 0.2 wt. % of Septon™ 1020 was required to increase the viscosity index of the hydrogenated C₃₂ PAO product from 107 to 120. Compared to Septon™ 1020 (Examples 16-17), Septon™ 2002 (Examples 18-19) was less effective at increasing the viscosity index of the hydrogenated C₃₂ PAO product. Additionally, the 100° C. kinematic viscosity (KV100), 40° C. kinematic viscosity (KV40), and −40° C. kinematic viscosity (KV-40) increased with addition of the copolymers but remained in viable ranges. Thus, hydrogenated styrenic block copolymers (Examples 16-19) were found to be effective additives to increase the viscosity index of the hydrogenated C₃₂ PAO product (Example 10) as needed.

TABLE I
Oligomerization of 2-hexyl-1-decene with
Various Catalysts-Examples 1 to 8

| Example | 1 | 2 | 3 | 4 | 5 | 6 | 7 | 8 |
|---|---|---|---|---|---|---|---|---|
| Catalyst | AlBr₃ | AlBr₃ | AlBr₃ | AlBr₃ | AlBr₃ | AlCl₃ | AlBr₃ | FeCl₃ |
| Cat. Loading (wt. %) | 7 | 0.8 | 0.3 | 1.1 | 1.1 | 3 | 0.01 | 4 |
| C₁₆ to C₃₂ Conversion | 80 | 77 | 76 | 74 | 80 | 80 | 1.5 | 10.6 |
| (wt. %) | | | | | | | | |
| C₃₂:C₄₈ wt. ratio | 4.0 | 4.0 | 5.0 | 4.3 | 3.6 | 5.2 | | —— |
| Time (h) | 2 | 3 | 1 | 1 | 1 | 1 | 1 | 24 |
| Temperature (° C.) | 100 | 100 | 100 | 130 | 70 | 100 | 100 | 25 |

TABLE II
Viscometric Properties of Hydrogenated Bottoms C₃₂ product-Batches 1 to 3

| Batch | 1 | 2 | 3 |
|---|---|---|---|
| 100° C. Kinematic | 5.7 | 4.35 | 4.91 |
| Viscosity, cSt | | | |
| 40° C. Kinematic Viscosity, | 31.7 | 21.1 | 25.1 |
| cSt | | | |
| Viscosity Index | 122 | 115 | 121 |

TABLE III
Physical Properties of Hydrogenated C₃₂ Oligomer Product

| Example | 10 | C11 | C12 | C13 | 20 |
|---|---|---|---|---|---|
| 100° C. Kinematic Viscosity, | 4.37 | 4.2 | 4.30 | 3.9 | 4.46 |
| cSt | | | | | |
| 40° C. Kinematic Viscosity, cSt | 21.99 | 18.3 | 20.1 | 16.8 | 22.87 |
| −40° C. Kinematic Viscosity, cSt | 3795 | No Flow | No Flow | 2,498 | Not tested |
| Viscosity Index | 107 | 133 | 121 | 124 | 107 |
| Pour Point (° C.) | −74 | −35 | −15 | −68 | −72 |
| Noack Volatility (%) | 12.8 | 14.1 | 15.0 | 13.4 | Not tested |
| Flash Point-Open Cup (° C.) | 238 | 231 | 230 | 226 | 250 |

TABLE IV
Investigation of Viscosity Modifiers-Metallocene Polyalphaolefins (mPAOs)

| Example | 10 | 14 | 15 |
|---|---|---|---|
| Modifier | None | mPAO 65 | mPAO 150 |
| Mod. Loading (wt. %) | 0 | 3 | 5 |
| 100° C. Kinematic | 4.37 | 4.67 | 5.12 |
| Viscosity, cSt | | | |
| Viscosity Index | 107 | 115 | 125 |

TABLE V
Investigation of Viscosity Modifiers-Hydrogenated Styrenic Block Copolymers

| Example | 10 | 16 | 17 | 18 | 19 |
|---|---|---|---|---|---|
| Modifier | None | Septon ™ | Septon ™ | Septon ™ | Septon ™ |
| | | 1020 | 1020 | 2002 | 2002 |
| Mod. Loading (wt. %) | 0 | 0.2 | 0.3 | 0.2 | 0.5 |
| 100° C. Kinematic | 4.37 | 4.74 | 4.85 | 4.73 | 4.96 |
| Viscosity, cSt | | | | | |
| 40° C. Kinematic | 21.99 | 23.78 | 24.38 | 24.09 | 25.36 |
| Viscosity, eSt | | | | | |
| −40° C. Kinematic | 3795 | 4870 | 5055 | 4796 | 5394 |
| Viscosity, cSt | | | | | |
| Viscosity Index | 107 | 120 | 123 | 116 | 122 |

The disclosure is described above with reference to numerous aspects and specific examples. Many variations will suggest themselves to those skilled in the art in light of the above detailed description. All such obvious variations are within the full intended scope of the appended claims. Other aspects of the disclosure can include, but are not limited to, the following (aspects are described as “comprising” but, alternatively, can “consist essentially of” or “consist of”):

Aspect 1. An oligomerization process comprising (i) contacting a first catalyst composition with an olefin feedstock comprising at least 50 wt. % (or at least 75 wt. %, at least 85 wt. %, at least 95 wt. %, at least 98 wt. %, or at least 99 wt. %) of 1-octene under first oligomerization conditions to produce a first oligomer product comprising a C₁₆ vinylidene dimer, (ii) separating (all or any portion of) the C₁₆ vinylidene dimer from the first oligomer product, and (iii) contacting (all or any portion of) the C₁₆ vinylidene dimer with a second catalyst composition under second oligomerization conditions to produce a second oligomer product comprising at least 50 wt. % (or at least 55 wt. %, at least 60 wt. %, at least 65 wt. %, at least 70 wt. %, or at least 75 wt. %) of C₃₂ olefins.

Aspect 2. A process to produce a polyalphaolefin comprising (I) contacting a first catalyst composition with an olefin feedstock comprising at least 50 wt. % (or at least 75 wt. %, at least 85 wt. %, at least 95 wt. %, at least 98 wt. %, or at least 99 wt. %) of 1-octene under first oligomerization conditions to produce a first oligomer product comprising a C₁₆ vinylidene dimer, (II) separating (all or any portion of) the C₁₆ vinylidene dimer from the first oligomer product, (III) contacting (all or any portion of) the C₁₆ vinylidene dimer with a second catalyst composition under second oligomerization conditions to produce a second oligomer product comprising at least 50 wt. % (or at least 55 wt. %, at least 60 wt. %, at least 65 wt. %, at least 70 wt. %, or at least 75 wt. %) of C₃₂ olefins, (IV) separating (all or any portion of) the C₃₂ olefins from the second oligomer product, and (V) hydrogenating (all or any portion of) the C₃₂ olefins to form the polyalphaolefin.

Aspect 3. The process defined in aspect 1 or 2, wherein the first catalyst composition comprises an organoaluminum compound and/or a metallocene compound.

Aspect 4. The process defined in aspect 3, wherein the organoaluminum compound comprises triisobutylaluminum (TIBA), trimethylaluminum (TMA), triethylaluminum (TEA), tri-n-propylaluminum (TNPA), tri-n-butylaluminum (TNBA), tri-n-hexylaluminum, tri-n-octylaluminum (TNOA), or combinations thereof.

Aspect 5. The process defined in any one of aspects 1-4, wherein a weight ratio of the first catalyst composition to the olefin feedstock (comprising the 1-octene) is in any range of weight ratios disclosed herein, e.g., from 1:1 to 1:100, from 1:5 to 1:50, from 1:10 to 1:50, or from 1:10 to 1:30.

Aspect 6. The process defined in any one of aspects 1-5, wherein the C₁₆ vinylidene dimer comprises 2-hexyl-1-decene.

Aspect 7. The process defined in any one of aspects 1-6, wherein the first oligomer product comprises any amount of the C₁₆ vinylidene dimer disclosed herein, e.g., at least 50 wt. %, at least 60 wt. %, at least 65 wt. %, at least 70 wt. %, at least 75 wt. %, at least 80 wt. %, or at least 85 wt. %.

Aspect 8. The process defined in any one of aspects 1-7, wherein separating (all or any portion of) the C₁₆ vinylidene dimer from the first oligomer product utilizes any technique disclosed herein, e.g., distillation, vacuum distillation, short path distillation, filtration, extraction, wiped film evaporation, or any combination thereof.

Aspect 9. The process defined in any one of aspects 1-8, wherein the first oligomer product further comprises unreacted olefin feedstock, tri-substituted dimer, and/or higher oligomers.

Aspect 10. The process defined in any one of aspects 1-9, wherein a conversion of the olefin feedstock (comprising the 1-octene) to the first oligomer product is in any range of conversions disclosed herein, e.g., from 40 wt. % to 95 wt. %, from 50 wt. % to 93 wt. %, from 60 wt. % to 91 wt. %, from 70 wt. % to 89 wt. %, or from 75 wt. % to 88 wt. %.

Aspect 11. The process defined in any one of aspects 1-10, further comprising a step of separating (all or any portion of) unreacted olefin feedstock from the first oligomer product using any technique disclosed herein, e.g., distillation, vacuum distillation, short path distillation, filtration, extraction, wiped film evaporation, or any combination thereof.

Aspect 12. The process defined in aspect 11, further comprising recycling (all or any portion of) the unreacted olefin feedstock to step (i) or step (I).

Aspect 13. The process defined in any one of aspects 1-12, further comprising a step of separating (all or any portion of) the first catalyst composition from the first oligomer product using any technique disclosed herein, e.g., distillation, vacuum distillation, short path distillation, filtration, extraction, wiped film evaporation, or any combination thereof.

Aspect 14. The process defined in aspect 13, further comprising recycling (all or any portion of) the separated first catalyst composition to step (i) or step (I).

Aspect 15. The process defined in any one of aspects 1-14, wherein the first catalyst composition and the second catalyst composition are different.

Aspect 16. The process defined in any one of aspects 1-15, wherein the second catalyst composition comprises an aluminum halide compound.

Aspect 17. The process defined in aspect 16, wherein the aluminum halide compound comprises aluminum bromide (AlBr₃) and/or aluminum chloride (AlCl₃).

Aspect 18. The process defined in any one of aspects 1-17, wherein a weight ratio of the second catalyst composition to the C₁₆ vinylidene dimer is in any range of weight ratios disclosed herein, e.g., from 1:1 to 1:2000, from 1:5 to 1:1000, from 1:10 to 1:500, or from 1:20 to 1:350.

Aspect 19. The process defined in any one of aspects 1-18, wherein the first oligomerization conditions and the second oligomerization conditions independently comprise an oligomerization temperature in any range disclosed herein, e.g., from 10° C. to 200° C., from 20° C. to 190° C., from 30° C. to 180° C., from 40° C. to 170° C., from 50° C. to 160° C., from 60° C. to 150° C., or from 70° C. to 140° C.

Aspect 20. The process defined in any one of aspects 1-19, wherein the second oligomer product further comprises any amount of C₄₈ olefins disclosed herein, e.g., less than or equal to 50 wt. %, less than or equal to 40 wt. %, less than or equal to 30 wt. %, less than or equal to 25 wt. %, or less than or equal to 20 wt. %.

Aspect 21. The process defined in any one of aspects 1-20, wherein a weight ratio of vinylidene dimers (C₃₂) to vinylidene trimers (C₄₈) in the second oligomer product is in any range disclosed herein, e.g., from 2:1 to 10:1, from 2:1 to 7:1, from 3:1 to 10:1, from 3:1 to 8:1, or from 3.5:1 to 6:1.

Aspect 22. The process defined in any one of aspects 1-21, wherein a conversion of the C₁₆ vinylidene dimer to the second oligomer product is in any range of conversions disclosed herein, e.g., from 40 wt. % to 95 wt. %, from 50 wt. % to 90 wt. %, from 60 wt. % to 87 wt. 00 from 70 wt. % to 85 wt. %, or from 73 wt. % to 83 wt. %.

Aspect 23. The process defined in any one of aspects 1-22, wherein the first oligomer product and the second oligomer product are produced, independently, in any oligomerization reactor disclosed herein, e.g., a fixed bed reactor, a stirred tank reactor, a plug flow reactor, a tubular reactor, or any combination thereof.

Aspect 24. The process defined in any one of aspects 1-23, further comprising a step of separating (all or any portion of) the second catalyst composition from the second oligomer product using any technique disclosed herein, e.g., distillation, vacuum distillation, short path distillation, filtration, extraction, wiped film evaporation, or any combination thereof.

Aspect 25. The process defined in aspect 24, further comprising recycling (all or any portion of) the separated second catalyst composition to step (iii) or step (III).

Aspect 26. The process defined in any one of aspects 2-25, wherein separating (all or any portion of) the C₃₂ olefins from the second oligomer product utilizes any technique disclosed herein, e.g., distillation, vacuum distillation, short path distillation, filtration, extraction, wiped film evaporation, or any combination thereof.

Aspect 27. The process defined in any one of aspects 2-26, wherein hydrogenating (all or any portion of) the C₃₂ olefins to form the polyalphaolefin utilizes any metal catalyst disclosed herein, e.g., containing platinum, rhenium, palladium, and/or nickel.

Aspect 28. The process defined in any one of aspects 2-27, wherein hydrogenating to form the polyalphaolefin is performed at a temperature in any range disclosed herein, e.g., from 10° C. to 200° C., from 20° C. to 180° C., from 50° C. to 160° C., or from 80° C. to 140° C.

Aspect 29. The process defined in any one of aspects 2-28, wherein the polyalphaolefin comprises at least 70 wt. % (or at least 72 wt. %, at least 75 wt. %, at least 77 wt. %, at least 78 wt. %, or at least 80 wt. %) C₃₂ alkanes (hydrogenated 1-octene tetramers).

Aspect 30. The process defined in aspect 29, wherein the C₃₂ alkanes comprise at least 25 wt. % (or at least 35 wt. %, at least 40 wt. %, at least 45 wt. %, at least 50 wt. %, or at least 60 wt. %) of a compound having the structure:

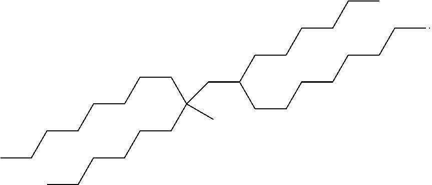 [SMILES: CCCCCCCCC(CCCCCC)CC(C)(CCCCCC)CCCCCCCC]

Aspect 31. The process defined in any one of aspects 2-30, wherein the polyalphaolefin is characterized by a 100° C. kinematic viscosity (KV100) in a range from 3.5 to 5 cSt, a pour point in a range from −90° C. to −50° C., a flash point in a range from 200° C. to 300° C., and/or a viscosity index in a range from 90 to 120.

Aspect 32. The polyalphaolefin produced by the process defined in any one of aspects 2-31.

Aspect 33. A polyalphaolefin characterized by a 100° C. kinematic viscosity (KV100) in a range from 3.5 to 5 cSt, a pour point in a range from −90° C. to −50° C., and a viscosity index in a range from 90 to 120.

Aspect 34. The polyalphaolefin defined in aspect 33, wherein the polyalphaolefin comprises at least 70 wt. % (or at least 72 wt. %, at least 75 wt. %, at least 77 wt. %, at least 78 wt. %, or at least 80 wt. %) of C₃₂ alkanes (hydrogenated 1-octene tetramers).

Aspect 35. A polyalphaolefin comprising at least 70 wt. % (or at least 72 wt. %, at least 75 wt. %, at least 77 wt. %, at least 78 wt. %, or at least 80 wt. %) C₃₂ alkanes (hydrogenated 1-octene tetramers) and characterized by a 100° C. kinematic viscosity (KV100) in a range from 3.5 to 5 cSt, a pour point in a range from −90° C. to −50° C., and a flash point in a range from 200° C. to 300° C.

Aspect 36. A polyalphaolefin comprising at least 70 wt. % (or at least 72 wt. %, at least 75 wt. %, at least 77 wt. %, at least 78 wt. %, or at least 80 wt. %) of C₃₂ alkanes (hydrogenated 1-octene tetramers) and characterized by a 100° C. kinematic viscosity (KV100) in a range from 3.5 to 5 cSt, a pour point in a range from −90° C. to −50° C., and a viscosity index in a range from 90 to 120.

Aspect 37. A polyalphaolefin comprising at least 70 wt. % (or at least 72 wt. %, at least 75 wt. %, at least 77 wt. %, at least 78 wt. %, or at least 80 wt. %) of C₃₂ alkanes (hydrogenated 1-octene tetramers) and characterized by a 100° C. kinematic viscosity (KV100) in a range from 3.5 to 5 cSt, and wherein the C₃₂ alkanes comprise at least 25 wt. % (or at least 35 wt. %, at least 40 wt. %, at least 45 wt. %, at least 50 wt. %, or at least 60 wt. %) of a compound having the structure:

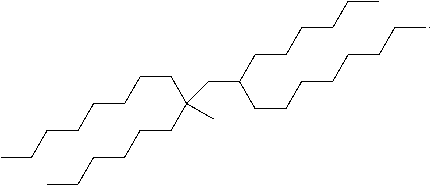 [SMILES: CCCCCCCCC(CCCCCC)CC(C)(CCCCCC)CCCCCCCC]

Aspect 38. The polyalphaolefin defined in any one of aspects 32-37, wherein the 100° C. kinematic viscosity (KV100) is in any range disclosed herein, e.g., from 3.7 to 4.8 cSt, from 3.7 to 4.6 cSt, from 3.9 to 4.6 cSt, from 4 to 4.5 cSt, from 4.2 to 4.8 cSt, or from 4.2 to 4.6 cSt.

Aspect 39. The polyalphaolefin defined in any one of aspects 32-38, wherein the pour point is in any range disclosed herein, e.g., from −90° C. to −50° C., from −85° C. to −65° C., from −85° C. to −70° C., or from −80° C. to −70° C.

Aspect 40. The polyalphaolefin defined in any one of aspects 32-39, wherein the viscosity index is in any range disclosed herein, e.g., from 90 to 120, from 95 to 115, from 100 to 115, or from 105 to 110.

Aspect 41. The polyalphaolefin defined in any one of aspects 32-40, wherein the polyalphaolefin or the C₃₂ alkanes comprise at least 25 wt. % (or at least 35 wt. %, at least 40 wt. %, at least 45 wt. %, at least 50 wt. %, or at least 60 wt. %) of a compound having the structure:

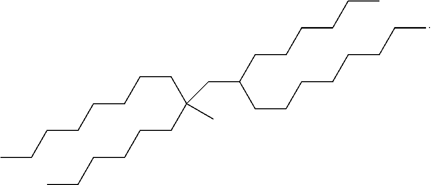 [SMILES: CCCCCCCCC(CCCCCC)CC(C)(CCCCCC)CCCCCCCC]

Aspect 42. The polyalphaolefin defined in any one of aspects 32-41, wherein the polyalphaolefin is further characterized by a flash point in any range disclosed herein, e.g., from 200° C. to 300° C., from 210° C. to 300° C., from 215° C. to 290° C., from 220° C. to 280° C., from 220° C. to 260° C., from 225° C. to 270° C., from 230° C. to 270° C., from 230° C. to 260° C., from 230° C. to 250° C., or from 235° C. to 240° C.

Aspect 43. The polyalphaolefin defined in any one of aspects 32-42, wherein the polyalphaolefin is further characterized by a 40° C. kinematic viscosity (KV40) in any range disclosed herein, e.g., from 5 to 50 cSt, from 10 to 40 cSt, from 15 to 30 cSt, or from 20 to 25 cSt.

Aspect 44. The polyalphaolefin defined in any one of aspects 32-43, wherein the polyalphaolefin is further characterized by a −40° C. kinematic viscosity (KV-40) in any range disclosed herein, e.g., from 2500 to 4500 cSt, from 3000 to 4500 cSt, from 3300 to 4300 cSt, from 3600 to 4000 cSt, or from 3700 to 3800 cSt.

Aspect 45. The polyalphaolefin defined in any one of aspects 32-44, wherein the polyalphaolefin is further characterized by a Noack volatility in any range disclosed herein, e.g., from 5% to 20%, from 7% to 15%, from 8% to 15%, from 10% to 14%, or from 11% to 13%.

Aspect 46. The polyalphaolefin defined in any one of aspects 33-45, wherein the polyalphaolefin is the polyalphaolefin produced by the process of any one of aspects 2-31.

Aspect 47. A lubricant composition comprising the polyalphaolefin defined in any one of aspects 32-46 and an additive.

Aspect 48. The lubricant composition defined in aspect 47, wherein the additive comprises an anti-wear additive, a dispersant, a viscosity modifier, a friction modifier/reducer, a detergent, a demulsifier, a defoamant, an antioxidant, an extreme pressure agent, a rust/corrosion inhibitor, a metal passivator, a pour point depressant, a thickener, or any combination thereof.

What is claimed is:

1. An oligomerization process comprising:
(i) contacting a first catalyst composition with an olefin feedstock comprising at least 50 wt. % of 1-octene under first oligomerization conditions to produce a first oligomer product comprising a C₁₆ vinylidene dimer;
(ii) separating the C₁₆ vinylidene dimer from the first oligomer product; and
(iii) contacting the C₁₆ vinylidene dimer with a second catalyst composition under second oligomerization conditions to produce a second oligomer product comprising at least 50 wt. % of C₃₂ olefins.

2. The process of claim 1, wherein:
the first catalyst composition comprises an organoaluminum compound and/or a metallocene compound;
a weight ratio of the first catalyst composition to the olefin feedstock is in a range from 1:1 to 1:100;
the first oligomer product comprises at least 50 wt. % of the C₁₆ vinylidene dimer; and
a conversion of the olefin feedstock to the first oligomer product is from 40 wt. % to 95 wt. %.

3. The process of claim 1, wherein:
the C₁₆ vinylidene dimer comprises 2-hexyl-1-decene; and
the first oligomer product further comprises unreacted olefin feedstock, tri-substituted dimer, and/or higher oligomers.

4. The process of claim 1, further comprising:
separating unreacted olefin feedstock from the first oligomer product and optionally recycling the unreacted olefin feedstock to step (i); and/or
separating the first catalyst composition from the first oligomer product and optionally recycling the separated first catalyst composition to step (i).

5. The process of claim 1, wherein:
the second catalyst composition comprises an aluminum halide compound;
a weight ratio of the second catalyst composition to the C₁₆ vinylidene dimer is from 1:1 to 1:2000; and
the first oligomerization conditions and the second oligomerization conditions independently comprise an oligomerization temperature from 10° C. to 200° C.

6. The process of claim 1, wherein:
the second oligomer product further comprises less than or equal to 40 wt. % of C₄₈ olefins;
a weight ratio of vinylidene dimers (C₃₂) to vinylidene trimers (C₄₈) in the second oligomer product is from 2:1 to 10:1;
a conversion of the C₁₆ vinylidene dimer to the second oligomer product is from 40 wt. % to 95 wt. %; or
any combination thereof.

7. The process of claim 1, further comprising a step of separating the second catalyst composition from the second oligomer product and optionally recycling the separated second catalyst composition to step (iii).

8. The process of claim 1, further comprising:
(iv) separating the C₃₂ olefins from the second oligomer product; and
(v) hydrogenating the C₃₂ olefins to form a polyalphaolefin.

9. The process of claim 8, wherein:
hydrogenating the C₃₂ olefins to form the polyalphaolefin utilizes a metal catalyst containing platinum, rhenium, palladium, and/or nickel;
hydrogenating to form the polyalphaolefin is performed at a temperature from 10° C. to 200° C.; and
the polyalphaolefin comprises at least 70 wt. % C₃₂ alkanes.

10. The process of claim 9, wherein the C₃₂ alkanes comprise at least 25 wt. % of a compound having the structure

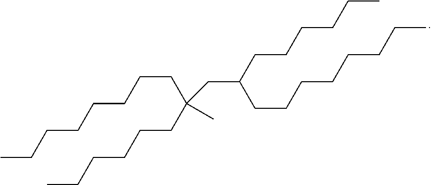 [SMILES: CCCCCCCCC(CCCCCC)CC(C)(CCCCCC)CCCCCCCC]

11. The process of claim 10, wherein the polyalphaolefin is characterized by:
a 100° C. kinematic viscosity (KV100) in a range from 3.5 to 5 cSt;
a pour point in a range from −90° C. to −50° C.;
a flash point in a range from 200° C. to 300° C.;
a viscosity index in a range from 90 to 120; or
any combination thereof.

12. A polyalphaolefin comprising at least 70 wt. % of C₃₂ alkanes and characterized by:
a 100° C. kinematic viscosity (KV100) in a range from 3.5 to 5 cSt;
a pour point in a range from −90° C. to −50° C.; and
a flash point in a range from 200° C. to 300° C.

13. The polyalphaolefin of claim 12, wherein the C₃₂ alkanes comprise at least 25 wt. % of a compound having the structure:

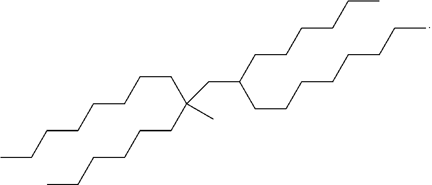 [SMILES: CCCCCCCCC(CCCCCC)CC(C)(CCCCCC)CCCCCCCC]

14. The polyalphaolefin of claim 12, wherein:
the 100° C. kinematic viscosity (KV100) is from 3.7 to 4.6 cSt;
the pour point is from −85° C. to −65° C.; and
the flash point is from 225° C. to 270° C.

15. The polyalphaolefin of claim 14, wherein:
the polyalphaolefin comprises at least 78 wt. % of the C₃₂ alkanes; and
the C₃₂ alkanes comprise at least 45 wt. % of a compound having the structure:

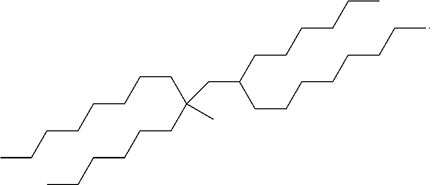 [SMILES: CCCCCCCCC(CCCCCC)CC(C)(CCCCCC)CCCCCCCC]

16. The polyalphaolefin of claim 12, wherein the polyalphaolefin is further characterized by a viscosity index from 90 to 120.

17. The polyalphaolefin of claim 12, wherein the polyalphaolefin is further characterized by:
a 40° C. kinematic viscosity (KV40) in a range from 10 to 40 cSt;
a −40° C. kinematic viscosity (KV-40) in a range from 3000 to 4500 cSt; and
a Noack volatility in a range from 7% to 15%.

18. A lubricant composition comprising the polyalphaolefin of claim 12 and an additive.

19. A polyalphaolefin characterized by:
a 100° C. kinematic viscosity (KV100) in a range from 3.5 to 5 cSt;
a pour point in a range from −90° C. to −50° C.; and
a viscosity index in a range from 90 to 120.

20. The polyalphaolefin of claim 19, wherein the polyalphaolefin comprises at least 70 wt. % of C₃₂ alkanes.
